SEQUENCE LISTING (14 sequences)

SEQ ID NO: 1 - DNA, 22 bp, Artificial sequence
  Synthetic primer
accacggtggagatagtgacaa

SEQ ID NO: 2 - DNA, 22 bp, Artificial sequence
  Synthetic primer
ggcttcttctcatttcctctgc

SEQ ID NO: 3 - DNA, 19 bp, Artificial sequence
  Synthetic primer
tggctgggagcataggagg

SEQ ID NO: 4 - DNA, 20 bp, Artificial sequence
  Synthetic primer
caggcaatgtagatggcggt

SEQ ID NO: 5 - DNA, 18 bp, Artificial sequence
  Synthetic primer
ccattcgaacgtctgccc

SEQ ID NO: 6 - DNA, 19 bp, Artificial sequence
  Synthetic primer
gtcacccgtggtcaccatg

SEQ ID NO: 7 - DNA, 23 bp, Artificial sequence
  Synthetic primer
gaaatgtcagttgctgcattcct

SEQ ID NO: 8 - DNA, 20 bp, Artificial sequence
  Synthetic primer
acaatccgcccaaagggaac

SEQ ID NO: 9 - DNA, 20 bp, Artificial sequence
  Synthetic primer
ctgctggcgtagcaggaagt

SEQ ID NO: 10 - DNA, 20 bp, Artificial sequence
  Synthetic primer
gctggaaagtgcctccattg

SEQ ID NO: 11 - DNA, 22 bp, Artificial sequence
  Synthetic primer
tcggcgagtctatgccactatt

SEQ ID NO: 12 - DNA, 24 bp, Artificial sequence
  Synthetic primer
acagagaacggatgagttgttcct

SEQ ID NO: 13 - DNA, 21 bp, Artificial sequence
  Synthetic primer
gatccattggagggcaagtct

SEQ ID NO: 14 - DNA, 28 bp, Artificial sequence
  Synthetic primer
aactgcagcaactttaatatacgctatt

(19) United States
(12) Patent Application Publication
(10) Pub. No.: US 2011/0136745 A1
(43) Pub. Date: Jun. 9, 2011

(54) METHOD OF SELECTING SOYBEANS WITH ENHANCED BIOACTIVITY AND COMPOSITIONS FOR REDUCING CANCER CELL VIABILITY

(75) Inventors: Neal Bringe, St. Charles, MO (US); Elvira De Mejia, Urbana, IL (US)

(73) Assignee: Monsanto Technology LLC.

(21) Appl. No.: 12/811,518

(22) PCT Filed: Dec. 31, 2008
(86) PCT No.: PCT/US2008/088647

§ 371 Date: Oct. 6, 2010

Related U.S. Application Data
(60) Provisional application No. 61/018,769, filed on Jan. 3, 2008.
(60) Provisional application No. 61/080,300, filed on Jul. 14, 2008.
(60) Provisional application No. 61/103,836, filed on Oct. 8, 2008.

(51) Int. Cl. A61K 38/14; G01N 33/53; G01N 33/574; C12Q 1/68; A01H 5/00; A01H 1/02; C07K 14/415; A61P 43/00; A61P 1/00; A23L 2/00; A23L 1/40; A21D 13/00; A23C 9/00; A23F 5/00; A23L 1/212; A23L 1/10

(52) U.S. Cl. 514/20.9; 435/7.1; 435/7.23; 435/7.21; 435/7.24; 435/6.17; 435/6.18; 800/312; 800/260; 530/378; 530/359; 426/656; 426/590; 426/589; 426/549; 426/580; 426/594; 426/634; 426/619; 426/599

(57) ABSTRACT

The invention provides a method that uses enzyme-treatment of whole soybeans or partially defatted soybeans to select soybeans with improved bioactivity or bioactivities. The invention further provides a soybean plant and seed with a non-transgenic mutation conferring enhanced bioactivity as an hydrolysate when compared to hydrolysate from other seeds, for instance in a cell-based assay, including reduced cancer cell viability; increased LDL receptor activity; reduced lipid accumulation; increased adiponectin expression; decreased FAS and LPL expression; reduced production of NO and PGE₂, and expression of iNOS and COX-2; higher antioxidant activity; promotion of growth of bifidobacteria; and inhibiting the growth of pathogenic bacteria; for instance when compared to other seeds tested as hydrolysates. The invention also provides soybean plants for use in producing seeds that have an overall improved bioactivity compared to other seeds as hydrolysates by combining effects on several bioactivity assays in a health index. The invention also provides products derived from, and parts of, these plants and uses thereof. Methods for producing such plants are also provided, as well as methods for standardizing or assuring quality control of soybean products with enhanced bioactivity for humans and animals.

### CROSS REFERENCE TO RELATED APPLICATIONS

This application claims the priority of U.S. Provisional Applications, Ser. Nos. 61/018,769, 61/080,300, and 61/103,836; filed Jan. 3, 2008, Jul. 14, 2008, and Oct. 8, 2008, respectively, the entire disclosures of which are incorporated herein by reference.

### INCORPORATION-BY-REFERENCE OF SEQUENCE LISTING IN COMPUTER READABLE FORM

The Sequence Listing, which is a part of the present disclosure, includes a computer readable form 3.00 KB file entitled “MONS212WO_Sequence_Listing” comprising nucleotide sequences of the present invention submitted via EFS-Web. The subject matter of the Sequence Listing is incorporated herein by reference in its entirety.

### BACKGROUND OF THE INVENTION

1. Field of the Invention

The present invention relates generally to the field of plant breeding. In particular, the invention relates to a method of selecting soybean varieties with improved bioactivities related to human health, for instance relating to improved or standardized anticancer, anti-obesity, prebiotic, antioxidant and/or anti-inflammatory bioactivities, as well as to food, drink, and topical compositions for improved health. Furthermore, the invention relates to agronomically elite soybean varieties with seed compositions having properties related to improved human health benefits such as anticancer, anti-obesity, prebiotic, antioxidant and anti-inflammatory bioactivity and materials for making such plants.

2. Description of Related Art

Soybeans grown for food use are typically sold in the form of roasted soybeans, fresh soybeans (edamame), defatted and whole bean flour, concentrates, isolates, purified protein fractions, peptides, soybean oil, fiber, isoflavones, phytosterols, tocopherols, and lecithins. The more refined soybean ingredients are valued because fractionation and concentration of targeted components and chemical, enzymatic and formulation treatments improve the taste and texture properties and enable product developers to have more flexibility in incorporating proteins and other fractions in foods. In an effort to create good tasting and formulation-friendly foods, it is often considered an improvement to remove astringent isoflavones, bitter saponins, poorly soluble fibers, flatulence producing oligosaccharides, oxidation prone phospholipids and bioactive amino acid sequences (e.g. proteins and peptides), such as protease inhibitors and lectins.

However, given the health-related properties beta-conglycinins, it is prudent to develop soybeans lacking glycinins and enriched in beta-conglycinins and examine the bioactivity of the new whole bean compositions. Thus, the present invention provides and includes a method to select soybean varieties wherein the whole or non-fat soybean is digested and examined for bioactivities related to human health. In addition, the present invention provides compositions of soybean varieties with improved human health bioactivities. Furthermore, the invention may be utilized for predicting the value of soybean varieties to make ingredients and foods for inhibiting cancer cells, lowering cholesterol and/or reducing inflammation. The invention may be further used to predict the value of soybean varieties to reduce body fat and health risks associated with excessive body fat.

### SUMMARY OF THE INVENTION

Methods and compositions are disclosed herein to obtain soybean varieties with improved bioactivity for human health, and products prepared therefrom. In one aspect, the present invention provides a method to evaluate the bioactivity of soybean varieties. In one embodiment, the invention may be used for selecting and developing improved soybean varieties. In a further embodiment, the method may be used to assure quality and consistency of the bioactivity of a soybean product, such as meal.

In some embodiments, the method contains steps that serve to mimic industrial processes and human and animal digestion. In certain embodiments, the method may comprise a step to extract fat from ground soybeans. Thus, soybeans may be ground in hot water (e.g. 80-95° C.) to simulate an industrial process, dried and defatted. The hydrated and ground soybeans may be modified with enzymes prior to drying and defatting to simulate an industrial product. In certain embodiments, methods of the invention include heat-treatments to inactivate bacteria and soybean and/or fungal enzymes, such as lipoxygenases, that may cause deterioration of food quality.

In some embodiments, the invention provides digestion conditions for modifying a soybean product, such as a meal, with a digestive enzyme, such as a protease. In various embodiments, the length of the period of digestion may range for from about 5 minutes to about 3 hours. In particular embodiments the digestion conditions comprise about 3 hours with pepsin followed by about 3 hours with pancreatin to produce bioactive peptides. Other enzymes, or enzyme preparations, such as Alcalase®, (Novo Nordisk A/S, Denmark); Alkaline Protease Concentrate (Valley Research, South Bend, Ind.); Protex™ 6L (Genencor, Palo Alto, Calif.), flavorzyme, trypsin, chymotrypsin and elastase may also be employed. Alternatively, enzymes including proteases can be provided by a bacterial culture such as a yogurt culture

Methods of the invention further provide bioactive peptides from soybeans that are produced by the action of proteases in the presence of soybean fiber, saponins and protease inhibitors. Furthermore the invention provides bioactive peptides that were produced from heat-treated dispersions of ground soybean or defatted soybean. Furthermore the invention provides bioactive peptides that maintain activity after further thermal treatments. In certain embodiments the thermal treatment or treatments model food processing conditions, and are conditions under which peptides of an hydrolysate are tested to determine whether they retained bioactivity. In particular embodiments bioactive peptides retains activity following such thermal treatment. The thermal treatment may be applied to a polypeptide, such as a protein, before, during, or after the soybean product, such as a meal, is contacted with a protease; that is, before peptide formation, during peptide formation, or after peptide formation. In some embodiments the peptide product is an article of commerce. The invention further provides methods for stopping or slowing the activity of the digestive enzymes. In particular embodiments, stopping or slowing the activity of the digestive enzymes may comprise heat treatment or treatment with trichloracetic acid (TCA). Alternative methods such as quick freezing may be used where the subsequent bioactivity assay is not affected by the presence of non-denatured digestive enzymes. In some embodiments a method of the invention comprises a centrifugation step to remove high molecular weight fibers and protein fractions that would not normally be present in vivo, e.g., at the liver. Alternatively, the centrifugation step can be omitted to study gut bioactivities such as bile acid binding or prebiotic or other effects on microflora populations.

Another aspect of the invention provides a method to phenotype the bioactivity of the whole soybean. In particular embodiments, the bioactive phenotype relates to a health benefit. In further embodiments, the health benefit is selected from the group consisting of antioxidant activity, anti-inflammatory activity, anti-fat accumulation activity (or fat burning activity), reduced cholesterol levels, reduced joint pain, reduced itching, prebiotic, antibiotic and anti-cancer activity. In certain embodiments the method to phenotype the bioactivity of a whole soybean allows for the development of one or more molecular markers. In particular embodiments, such a marker is used for introgressing a genetic trait that specifies such bioactivity into soybean germplasm. This utility is also useful for identifying genetic targets for additional improvements. The utility of the invention is further demonstrated by determining bioactivities of soybean hydrolysates and correlating the compositions of the soybeans and hydrolysates prepared therefrom.

The present invention also relates to an elevated β-conglycinin composition of soybean seed which has improved bioactivity related to human and animal health, such as reduced cancer cell viability, increased cholesterol metabolism, reduced lipid accumulation, or increased adiponectin expression compared with commercial soybean protein ingredients. The current invention further provides a soybean plant with non-transgenic traits conferring elevated β-conglycinin composition of soybean seed. In certain embodiments, the seed β-conglycinin content for plants of the invention is about or at least about 27, 28, 29, 30, 31, 32, 33, 34, 35, 36, 37, 38, 39, 40, 41, 42, 43, 44, 45, 46, 47, 48, 49, 50, 51, 52, 53, 54 percent or more of the total protein content. In some embodiments, the seed glycinin content for plants of the invention is less than 30 percent of the total protein content.

Plant parts are also provided by the invention. Parts of a plant of the invention include, but are not limited to, pollen, ovules, meristems, cells, and seed. Cells of the invention may further comprise, regenerable cells, such as embryos meristematic cells, pollen, leaves, roots, root tips, and flowers. Thus, these cells could be used to regenerate plants of the invention.

Also provided herein are parts of the seeds of a plant according to the invention. Thus, crushed seed, and meal or flour made from seed according to the invention is also provided as part of the invention. The invention further comprises a method for making soy meal or flour comprising crushing or grinding seed according to the invention. Such soy flour or meal according to the invention may comprise genomic material of plants of the invention. In one embodiment, the food may be defined as comprising the genome of such a plant. In further embodiments soy meal or flour of the invention may be defined as comprising increased β-conglycinin and decreased glycinin content, as compared to meal or flour made form seeds of a plant with an identical genetic background, but not comprising the non-transgenic, mutant Gy3 and Gy4 null alleles.

Another aspect of the invention provides a method for inhibiting inflammation, pain or itching, comprising: applying to a subject a topical composition comprising β-conglycinin or a protease-treated preparation of β-conglycinin. In certain embodiments the composition comprises: (a) a protease-treated preparation made from soybean seed wherein at least 27% of the total protein in the soybean seed comprises β-conglycinins and less than 30% of the total protein in the soybean seed comprises glycinins; and (b) a cosmetically acceptable topical carrier.

The invention further provides a method for preventing or inhibiting inflammation and/or associated pain, comprising: consuming a food or drink made from soybean seed wherein at least 27% of the total protein in the soybean seed comprises β-conglycinins and less than 30% of the total protein in the soybean seed comprises glycinins. In certain embodiments, the food or drink is selected from the group consisting of: a liquid form of edamame; a powder form of edamame; roasted soybeans; soymilk; soy kefir; soy yogurt; soy flour; soy protein concentrate; soy protein isolate, and β-conglycinin isolate. In particular embodiments the soymilk is whole bean soymilk. In other embodiments the soymilk is treated for over one hour with a protease. In certain embodiments the protease is Alcalase®. In yet other embodiments, consumption follows physical exercise or participation in a sporting competition. In some embodiments, the β-conglycinin or β-conglycinin peptides is (are) administered to a subject in the amount of about 1 gram to about 50 grams per day.

Yet another aspect of the invention provides a topical composition comprising β-conglycinin or a protease-treated preparation of β-conglycinin. In certain embodiments the composition comprises: (a) a protease-treated preparation made from soybean seed wherein at least 27% of the total protein in the soybean seed comprises β-conglycinins and less than 30% of the total protein in the soybean seed comprises glycinins; and (b) a cosmetically acceptable topical carrier. In further embodiments, the β-conglycinin is prepared from soybean seed wherein greater than 33% of the total soy protein or peptides originate from β-conglycinins.

Yet another aspect of the invention provides a method of testing a human food for improved or standardized bioactivity associated with human health, wherein the food comprises soybeans and at least one other plant protein source, and: (a) ground soybeans and the at least one additional plant protein source are heat-treated; (b) the heat treated preparation of step (a) is digested with one or more proteases to form a digest or an extract of the digest; and (c) the digest or extract of the digest is included in a bioactivity assay. Parameters for digestion with the one or more proteases may be varied, for instance, to reduce formation of peptides that contribute to off-flavors. In certain embodiments, the plant protein source or sources is selected from the group consisting of: sunflower seeds, flax seeds, sesame seeds, and a vegetable, a nut or a bean, or other vegetable. In particular embodiments, the vegetable is spinach, broccoli, tomatoes, peppers, lettuce, purple carrot, or cucumber. Example of foods with protein blends are broccoli soup containing soymilk, chopped spinach in soymilk sauce, bean burrito filling containing soymilk powder, soymilk beverage containing lettuce, carrot or cucumber juice or almond milk or sesame seed milk, a bakery product containing soymilk powder and sunflower seeds or flax seed flour, or broccoli.

In yet a further aspect of the invention there is provided a method for producing a soybean seed, comprising crossing the plant of the invention with itself or with a second soybean plant. Thus, this method may comprise preparing a hybrid soybean seed by crossing a plant of the invention with a second, distinct, soybean plant.

Still yet another aspect of the invention is a method of producing a food product for human or animal consumption comprising: (a) obtaining a plant of the invention; (b) cultivating the plant to maturity; and (c) preparing a food product from the plant. In certain embodiments of the invention, the food product may comprise protein concentrate, protein isolate, meal, flour or soybean hulls. In some embodiments, the food product may be selected from the group consisting of: a beverage, an infused food, sauce, coffee creamer, a cookie, an emulsifying agent, bread, candy, an instant milk drink, gravy, noodles, soynut butter, soy coffee, roasted soybeans, crackers, soymilk, tofu, tempeh, baked soybeans, a bakery ingredient, a beverage powder, a breakfast cereal, a nutritional bar, meat or a meat analog, fruit juice, a dessert, a soft frozen product, a confection, and an intermediate food. Foods produced from the plants of the invention may comprise elevated β-conglycinin and thus be of greater nutritional value foods made with typical soybean varieties

A further aspect of the invention is a method of producing a nutraceutical, comprising: (a) obtaining a plant of the invention; (b) cultivating the plant to maturity; and (c) preparing a nutraceutical from the plant. Products produced from the plants of the invention may comprise elevated β-conglycinin content and thus be of greater nutritional value foods made with typical soybean varieties. For example, products from soybean seeds with elevated β-conglycinin may be used alone or combination with other mechanisms in a lipid-lowering therapy.

In further embodiments, a plant of the invention may further comprise a transgene. The transgene may in one embodiment be defined as conferring preferred property to the soybean plant selected from the group consisting of herbicide tolerance, increased yield, insect control, fungal disease resistance, virus resistance, nematode resistance, bacterial disease resistance, mycoplasma disease resistance, altered fatty acid composition, altered oil production, altered amino acid composition, altered protein production, increased protein production, altered carbohydrate production, germination and seedling growth control, enhanced animal and human nutrition, low raffinose, drought and/or environmental stress tolerance, altered morphological characteristics, increased digestibility, industrial enzymes, pharmaceutical proteins, peptides and small molecules, improved processing traits, improved flavor, nitrogen fixation, hybrid seed production, reduced allergenicity, biopolymers, biofuels, or any combination of these.

In certain embodiments, a plant of the invention may be defined as prepared by a method wherein a plant comprising non-transgenic mutations conferring enhanced bioactivity associated with human and animal health is crossed with a plant comprising agronomically elite characteristics and enhanced human health properties. The progeny of this cross may be assayed for agronomically elite characteristics, bioactivity, and progeny plants selected based on these characteristics, thereby generating the plant of the invention. Thus in certain embodiments, a plant of the invention may be produced by crossing a selected starting variety with a second soybean plant comprising agronomically elite characteristics.

The present invention provides methods for producing in soybean plants with enhanced human health properties, such as reduced cancer cell viability, increased cholesterol metabolism, reduced lipid accumulation, or increased adiponectin expression compared to controls (commodity soybeans, commercial soybean protein ingredients and alternative food protein sources). The present invention relates to methods to determine the presence or absence of quantitative trait loci conferring enhanced human health prosperities in soybean plants, including but not limited to exotic germplasm, populations, lines, elite lines, cultivars and varieties. The present invention is not limited to any type of human health trait, including, but not limited to reducing cancer cell viability, increasing cholesterol metabolism, reducing lipid accumulation, and increasing adiponectin expression. More particularly, the invention relates to methods involving for identifying molecular markers associated with human health properties quantitative trait loci (QTL). The present invention relates to the use of molecular markers to screen and select for human health properties within soybean plants, including but not limited to exotic germplasm, populations, lines, elite lines, and varieties.

Embodiments discussed in the context of a method and/or composition of the invention may be employed with respect to any other method or composition described herein. Thus, an embodiment pertaining to one method or composition may be applied to other methods and compositions of the invention as well.

As used in the specification or claims, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1: Percent cell viability of L1210 leukemia cells after treatment for 48 h with 6 mg/ml of soy hydrolysates (NBH1-NBH7). L1210 leukemia cells were cultured at 37° C. in a 5% humidified CO₂ atmosphere in Minimum Essential Medium supplemented with 10% bovine serum. A Cell Counting Kit-8 was used to determine the number of viable cells. The results are expressed as percent viability of treated cells compared with the untreated control. NBHs with different letters were significant different (p<0.05, one way ANOVA with Tukey's multiple comparison test). Positive control, etoposide, inhibited 90.5% of L1210 leukemia cell growth at 1.0 μM level.

FIG. 2: Comparison of cytotoxicity of glycinin and β-conglycinin on L1210 leukemia cells. (A) Comparison between average IC₅₀ values of NBHs derived from proteins containing more than 50% glycinin (NBH1, NBH2 and NBH7) and less than 50% glycinin (NB3, NB4, NB5 and NB6). A significant difference between these two groups was observed based on unpaired t-test; (B) Dose dependent percent cell viability of purified glycinin and β-conglycinin on L1210 leukemia cells. Glycinin and β-conglycinin were purified from defatted soy flour and subject to sequential pepsin-pancreatin digestion. The hydrolysates were tested on log phase L1210 leukemia cells for 48 h. A Cell Counting Kit-8 was used to determine the number of viable cells. The results are expressed as percent viability of treated cells compared with the untreated control. Significant difference between glycinin and β conglycinin hydrolysate at certain concentration was indicated (*, p<0.05; **, p<0.01; one way ANOVA with Tukey's multiple comparison test).

FIG. 3: Relative mRNA expression of the LDL-receptor to two housekeeping genes presented as the mean of two runs, each incubated with HepG2 cells in triplicate. For NB HBC1 only one run was tested on cells in triplicate. The mean of NB cont-1 and NBcont 2 was set on 100%. The results of NB-HBC2 and NB-HBC3 were combined and presented in one bar.

FIG. 4: Relative mRNA expression of HMG-CoA reductase two housekeeping genes presented as the mean of two runs, each incubated with HepG2 cells in triplicate. For NB HBC1 only one run was tested on cells in triplicate. The mean of NB cont-1 and NB cont-2 was set on 100%. The results of NB-HBC2 and NB-HBC 3 were combined and presented in one bar.

FIG. 5: (A) Effect of soy Alcalase® hydrolysates (SH1-SH15) on inhibition of lipid accumulation (%) vs. control in 3T3-L1 adipocytes. 3T3-L1 adipocytes were harvested 8 days after the initiation of differentiation. Cells were treated with 100 μM soy hydrolysates for 72 h at 37° C. in a humidified 5% CO₂ incubator. (B) Comparison between average inhibition of lipid accumulation (%) values of milk alcalase hydrolysate and SHs derived from proteins containing 24.7+/−1.5% β-conglycinin (S1, S6, S7, S8 and S9) and 45.3+/−3.3 T % β-conglycinin (S2-S5 and S10-S15). Error bars indicate the standard deviation. Different letters indicate significant difference, p<0.0001. (C) Correlation between % inhibition of lipid accumulation and total β-conglycinin content of soy alcalase hydrolysates.

FIG. 6: Correlation between experimental and predicted inhibition of lipid accumulation values of soy hydrolysates on 3T3-L1 adipocytes. A partial least squares model was build to calculate predicted lipid accumulation inhibition values from the protein distribution (% total protein) in soy hydrolysates.

FIG. 7: Dose-dependent inhibition of lipid accumulation (%) vs. control in 3T3-L1 adipocytes after 72 h exposure to pure β-conglycinin and glycinin alcalase hydrolysate.

FIG. 8: Induction of adiponectin levels in 3T3-L1 adipocytes after treatment for 24 h with 100 μM of soy protein hydrolysates (SH1-SH15). (A) Fold increase in high molecular weight (HMW) adiponectin (60 KDa); and (B) Low molecular weight (LMW) adiponectin (30 KDa) of SHs compared to their controls. The relative expression of adiponectin in 3T3-L1 adipocytes was quantified densitometrically and calculated according to the references bands of β-actin.

FIG. 9: Dose-dependent induction of HMW and LMW adiponectin in 3T3-L1 adipocytes after 24 h treatment with pure β-conglycinin and glycinin alcalase hydrolysates.

FIG. 10: Effect of SAH (100 μM) on LPL (A) and FAS (B) mRNA abundance relative to negative control (untreated cells) in 3T3-L1 adipocytes after 72 h. Means with different letters are significantly different (P<0.0001, n=3 for LPL and FAS mRNA abundance). Bars indicate standard deviation.

FIG. 11: Single linear correlations of (A) lipid accumulation inhibition with FAS mRNA expression; (B) lipid accumulation inhibition with LPL mRNA expression; (C) β-conglycinin (% total protein) with FAS mRNA expression; and (D) β-conglycinin (% total protein) with LPL mRNA expression of SAH.

FIG. 12: Effect of SGIH (100 μM) on LPL (A) and FAS (B) mRNA abundance relative to negative control (untreated cells) in 3T3-L1 adipocytes after 72 h. Means with different letters are significantly different (P<0.05, n=3 for LPL mRNA abundance and P>0.05, n=3 for FAS mRNA abundance). Bars indicate standard deviation.

FIG. 13: Effect of SAH (100 μM), SAH followed by simulated GI digestion and SGIH on LPL and FAS gene expression in 3T3-L1 adipocytes after 72 h. Means with different letters are significantly different (P=0.0106, n=3 for LPL gene expression and P<0.0001, n=3 for FAS gene expression).

FIG. 14: Effect of different concentrations of SAH (D1 and D2) in nitrite production (A) and iNOS protein expression (B) by LPS-stimulated RAW 264.7 macrophage cell. Means with different letters are significantly different from the positive control (P<0.0001, n=3 for nitrite production; and P<0.0001, n=2 for iNOS expression).

FIG. 15: Effect of different concentrations of SAH (D1 and D2) in PGE₂ production (A) and COX-2 protein expression (B) by LPS-stimulated RAW 264.7 macrophages. Means with different letters are significantly different from the positive control (P<0.0001, n=3 for PGE₂ production, and P=0.0001, n=2 for COX-2 protein expression).

FIG. 16: Microarray intensity data corresponding to selected bifidobacteria after exposure (24 hr) of the tester flora to the soy hydrolysates (M-A1, M-A2, M-A3, M-B1, M-B2, M-C1, M-C2, M-D1, and M-D2), a positive control (GOS) and a negative control (water). Exposure to GOS resulted in a significant increased Bifidobacterial microarray signal in comparison to the water control, indicating a higher abundance. Soy hydrolysates of M-A1 and M-A3, M-B1, M-B2 and M-D1 stimulated B. longum, B. catenulatum and B. bifidum. The absolute height is dependent on the natural abundance of the species, behaviour in the flora and microarray probe characteristics.

FIG. 17: A summary of microarray intensity data corresponding to Enterobacteriaceae, including Salmonella, after exposure (5-48 hr) of the tester flora to the soy fractions, a positive control (GOS) and a negative control (water).

FIG. 18: Microarray intensity data corresponding to Enterobacteriaceae, including Salmonella, after exposure (24 hr) of the tester flora to the soy hydrolysates (M-A1, M-A2, M-B1, M-B2, M-C1, M-C2, M-D1, M-D2), a positive control (GOS), or a negative control (water).

### DESCRIPTION OF ILLUSTRATIVE EMBODIMENTS

The present invention provides plants and methods for producing plants comprising non-transgenic mutations that confer enhanced bioactivity for human and animal health. In particular, seed with elevated β-conglycinin reduce the cell viability of cancer cells. Thus, plants of the invention are of great value as enhanced bioactivity may provide enhanced nutritional characteristics of the soybean flour and protein isolates.

A limitation of methods of testing whole foods is that the compositions of food extracts (e.g., alcohol extract or filtered juice) typically lack digested proteins and associated bioactive peptides, and thus the value of bioactive peptides found in whole foods may not be recognized. The potential value of bioactive peptides released from intact proteins of whole foods during digestion is especially significant for soybeans which have a high content of protein (40%, dry basis). Methods were developed to simulate human digestion (U.S. Pat. No. 5,525,305) and have been used to compare the bioactivities of purified ingredients such as different types of inulins (van Nuenen et al., 2003). The digestion models have also been proposed for use in assessing the quality of food proteins according to their digestibility and content of amino acids (Schaafsma, 2005). A sunflower protein isolate was digested sequentially with pepsin and pancreatin enzymes as a first step to identify peptides with ACE inhibitor activity (Megias et al., 2004). Human and animal studies are costly, take a long time, and therefore are not very practical for making soybean variety selections.

Soybeans are associated with numerous benefits associated with cardiovascular health, weight management and healthy, non-cancerous cells. Several nutritional intervention studies in animals and humans indicate that consumption of soy protein reduces body weight and fat mass in addition to lowering plasma cholesterol and triglycerides. In animal models of obesity, soy protein ingestion limits or reduces body fat accumulation and improves insulin resistance (Hurley et al., 1998). In obese humans, dietary soy protein also reduces body weight and body fat mass in addition to reducing plasma lipids (Anderson et al., 2004).

Accumulation of body fat arises from a chronic imbalance between energy acquisition and expenditure that may lead to a pathologic growth of adipocytes, characterized by increased fat cell size and number (Shimomura et al., 1998). In addition, high body fat levels are closely associated with a state of chronic low-grade inflammation characterized by abnormal cytokine production and activation of inflammatory signaling pathways in adipose tissue (Hotamisligil, 2006). Adipose tissue can be regulated by the inhibition of adipogenesis and fat deposition (Rahman et al., 2008). Fat deposition in adipose tissue can be reduced by reducing lipid uptake by adipocytes via suppressing lipoprotein lipase (LPL) or reducing lipid synthesis through inhibiting fatty acid synthase (FAS) among other mechanisms (Jing-Jing et al., 2008). FAS inhibitors may be a target enzyme for obesity therapy (Kuhajda et al., 2005).

Adipose tissue macrophages accumulated during diet-induced accumulation of body fat may cause adipose tissue inflammation, alter insulin sensitivity and promote atherosclerosis (van Gaal et al., 2006; Weisberg et al., 2006). Macrophages secrete pro-inflammatory responses such as nitric oxide (NO) and prostaglandin E₂ (PGE₂). NO is produced by inducible NO synthase (iNOS) in activated macrophages and is an important inflammatory mediator (Guha and Mackman, 2001). In addition, cyclooxygenase-2 (COX-2) catalyzes the production of prostaglandins during the inflammatory process.

The properties and mechanisms associated with these benefits are also numerous and involve additive and synergistic effects, limiting the number and amount of soybeans and other whole foods that one needs to consumed for optimum health. In contrast, purified components of soybeans like phytosterols, may act by only one or two mechanisms and primarily affect one bioactivity, such as cholesterol absorption, and therefore have limited impact on overall health.

Thus it would be useful to be able to test whole soybean compositions for bioactivities, recognizing that a whole soybean that has one improved bioactivity will also continue to have multiple other bioactivities that are beneficial to health. In certain embodiments whole soybean compositions may be tested in multiple bioactivity assays to select varieties that generally optimize benefits.

Purified soybean components may be tested for bioactivity and for marketing the ingredients as supplements to create healthier versions of water, bread, cereals, bars and nutritional beverages. To identify a potentially marketable bioactive peptide, soy protein isolate is hydrolyzed by industrial enzymes and purified fractions using gel filtration chromatography and HPLC are tested for bioactivity (Lee & Kim, 2004). Isoflavones and saponins or mixtures can be extracted using alcohol and concentrated and further refined to created bioactive products (U.S. Pat. No. 6,607,757).

The amount and quality of digestive releases of bioactive amino acid sequences depend on the amount of protein containing the bioactive sequences and how other components affect their digestion and absorption (Shimoyamada et al., 1998). Compositional differences that could alter the amount of bioactive peptides include, for example, the ratio of protein fractions and variations in subunit concentrations within fractions. Soy proteins have four major water-extractable fractions (2S, 7S, 11S, and 15S) that can be isolated on the basis of their sedimentation coefficients. The 7S (β-conglycinin) and 11S (glycinin) proteins represent the majority of the fractions within the soybean. Other proteins with bioactivities include lunasin, Kunitz trypsin inhibitor and Bowman Birk Inhibitor.

The glycinin (11s globulin) is composed of five different subunits, designated A1aB2, A2B1a, A1bB1b, A5A4B3, A3B4, respectively. Each subunit is composed of two polypeptides, one acidic and one basic, covalently linked through a disulfide bond. The two polypeptide chains result from post-translational cleavage of proglycinin precursors; a step that occurs after the precursor enters the protein bodies (Chrispeels et al., 1982). Five major genes have been identified to encode these polypeptide subunits. They are designated as gy1, gy2, gy3, gy4 and gy5, respectively (Nielsen et al., 1997). In addition, a pseudogene, gy6, and minor gene, gy7, were also reported (Beilinson et al., 2002). Genetic mapping of these genes has been reported by various groups (Diers et al., 1993, Chen and Shoemaker 1998, Beilinson et al., 2002). Gy1 and gy2 were located 3 kb apart and mapped to linkage group N (Nielsen et al., 1989), gy3 was mapped to linkage group L (Beilinson et al., 2002). gy4 and gy5 were mapped to linkage groups O and F, respectively.

β-conglycinin (7S) is composed of a (˜67 kDa), α′ (˜71 kDa) and β (˜50 kDa) subunits and each subunit is processed by co- and post-translational modifications (Ladin et al., 1987; Utsumi, 1992). The α, and α′ subunits consist of core regions with high degree of homology (86.8%) and extension regions (α, 125 residues; α′, 141 residues) exhibiting lower homologies (57.3%), whereas the β subunit only consists of a core region that has homology with the α and α′ core regions (75.5%, 71.4%, respectively) (Utsumi, 1992). The β-conglycinin subunits are encoded by the genes cgy1, cgy2 and cgy3, respectively. Genetic analysis indicated that cgy2 is tightly linked to cgy3, whereas cgy1 segregates independently of the other two. The β-conglycinin gene family contains at least 15 members divided into two major groups, which encode the 2.5 kb and 1.7 kb embryo mRNA, respectively (Harada et al., 1989). The relative percentages of α′, α, and β chains in the trimer are ˜35, 45, and 20% of total β-conglycinin, respectively (Maruyama et al., 1999).

β-conglycinin has significant potential to positively impact human health (Baba et al., 2004). In particular, β-conglycinin has been found to lower cholesterol, triglycerides and visceral fat. Kohno et al. demonstrated that a significant reduction in triglycerol levels and visceral fat in human subjects that consumed 5 g of β-conglycinin per day (Kohno et al. 2006). Similarly, Nakamura et al. found that β-conglycinin up regulates genes associated with lipid metabolism in a primate model (2005). In addition, Nakamura et al. showed β-conglycinin had a significant effect preventing bone mineral density loss (2006). In addition, β-conglycinin demonstrated effects in lowering serum insulin and blood sugar (Moriyama et al. 2005). Due to β-conglycinin effects on triglycerides, cholesterol, fat, insulin and sugar levels, it may play an important role in health programs. In addition, β-conglycinin inhibits artery plaque formation in mice and may have similar affects in human subjects as well (Adams et al. 2004).

Furthermore, β-conglycinin may have a significant effect on intestinal microflora in humans. β-conglycinin is inhibits growth of harmful bacteria, such as E. coli, while stimulating growth of beneficial bacteria, such as Bifidobacteria, in a number of animal models (Nakamura et al. 2004, Zou et al. 2005,). β-conglycinin could be used both to reduce E. coli growth after infection and maintain a healthy intestinal microbial community. A hamster study supported the hypothesis that soy protein isolates made from high beta-conglycinin soybeans may have improved cholesterol and triglyceride lowering properties (Bringe, 2001). There are reports on the mechanisms and actions of lunasin and Bowman Birk inhibitor to inhibit cancer cells (Kennedy et al. 1996, Galvez 2001), but there are no reports of their relative important within an unrefined soybean ingredient.

The α′ subunit of β-conglycinin may play a predominant role in many of the health benefits associated with β-conglycinin A number of experiments using animal models have indicated that α′ subunit from soybean β-conglycinin could lower plasma triglycerides, and also increase LDL (“bad” cholesterol) removal from blood (Duranti et al., 2004, Moriyama et al., 2004, Adams et al., 2004, Nishi et al., 2003). Therefore, soybean varieties with an increased β-conglycinin content will have higher value than traditional varieties and will be suitable for use in nutrition drinks and other food products. In an attempt to identify the biologically active polypeptide(s), Manzoni et al. attempted to characterize biologically active polypeptides in β-conglycinin and indirectly demonstrated that the α′-subunit had a putative role in lowering cholesterol (Manzoni et al., 1998). Additionally, Manzoni et al. also demonstrated the influence of the α′ subunit on the increase in LDL uptake and degradation and LDL receptor mRNA levels (Manzoni et al., 2003). Duranti et al. (2004) demonstrated that the α′ subunit can lower triglycerides and plasma cholesterol in vivo.

The β-subunit of β-conglycinin has a number of properties related to health benefits as well. For instance, the β-subunit enhances satiety by causing cholecystokinin secretion (Takashi et al., 2003, Hara et al. 2004). Cholecystokinin is a peptide hormone of the gastrointestinal system responsible for stimulating the digestion of fat and protein. Cholecystokinin, previously called is synthesized by I-cells and secreted in the duodenum, the first segment of the small intestine, and causes the release of digestive enzymes and bile from the pancreas and gallbladder, respectively. It also acts as a hunger suppressant. Hence, β-subunit may suppress appetite and may play a role in an overall weight management program.

The β-subunit may have a function in reducing mental stress as well. Soymorphin-5 are released by digesting the β-subunit with pancreatic elastase and leucine aminopeptidase. Soymorphin-5 is an opioid peptide. Opioids are chemical substances that have a morphine-like action in the body. Opioids are primarily used for pain relief. These agents work by binding to opioid receptors, which are found principally in the central nervous system and the gastrointestinal tract. Soymorphin-5 demonstrated anxiolytic effect after oral administration on mice, which suggest the intake of β-subunit may decrease mental stress (Agui et al. 2005).

### I. PLANTS OF THE INVENTION

The invention provides, for the first time, plants and derivatives thereof of soybean that combine non-transgenic mutations conferring enhanced bioactivity for human and animal health. In certain embodiments, the β-conglycinin content of the seeds of plants of the invention may be greater than about 27, 28, 29, 30, 31, 32, 33, 34, 35, 36, 37, 38, 39, 40, 41, 42, 43, 44, 45, 46, 47, 48, 49, 50, 51, 52, 53, 54 percent or more of the total protein content.

One aspect of the current invention is therefore directed to the aforementioned plants and parts thereof and methods for using these plants and plant parts. Plant parts include, but are not limited to, pollen, an ovule and a cell. The invention further provides tissue cultures of regenerable cells of these plants, which cultures regenerate soybean plants capable of expressing all the physiological and morphological characteristics of the starting variety. Such regenerable cells may include embryos, meristematic cells, pollen, leaves, roots, root tips or flowers, or protoplasts or callus derived therefrom. Also provided by the invention are soybean plants regenerated from such a tissue culture, wherein the plants are capable of expressing all the physiological and morphological characteristics of the starting plant variety from which the regenerable cells were obtained.

### II. PRODUCTION OF SOYBEAN VARIETIES WITH ENHANCED BIOACTIVITY RELATED TO HUMAN AND ANIMAL HEALTH

The present invention describes methods to produce soybean plants with enhanced bioactivity related to human and animal health. Certain aspects of the invention also provide methods for selecting parents for breeding of plants with enhanced bioactivity related to human and animal health. One method involves screening germplasm for bioactivity of the whole soybean seed. More practical information can be obtained by examining the effects of a whole food, like soybean meal, than by examining effects of isolated food components, like isoflavonoids, on human and animal systems. The process involves simulating the industrial processing to inactivate soybean components that would be denatured during processing. The method also involves simulating the digestion process in humans and animals to activate bioactive protein. The bioactivities of human and/or animal cells are evaluated after being subjected to the bioactive peptides.

Certain aspects of the invention also provide methods for breeding of plants that enable the introduction of non-transgenic enhanced bioactivity for human and animal health traits into a heterologous soybean genetic background. In general, breeding techniques take advantage of a plant's method of pollination. There are two general methods of pollination: self-pollination which occurs if pollen from one flower is transferred to the same or another flower of the same plant, and cross-pollination which occurs if pollen comes to it from a flower on a different plant. Plants that have been self-pollinated and selected for type over many generations become homozygous at almost all gene loci and produce a uniform population of true breeding progeny, homozygous plants.

In development of suitable varieties, pedigree breeding may be used. The pedigree breeding method for specific traits involves crossing two genotypes. Each genotype can have one or more desirable characteristics lacking in the other; or, each genotype can complement the other. If the two original parental genotypes do not provide all of the desired characteristics, other genotypes can be included in the breeding population. Two parents which possess favorable, complementary traits are crossed to produce an F₁. An F₂ population is produced by selfing one or several F₁'s. Selection of the best individuals may begin in the F₂ population (or later depending on the breeder's objectives); then, beginning in the F₃, the best individuals in the best families can be selected. Replicated testing of families can begin in the F₃ and F₄ generation to improve the effectiveness of selection for traits with low heritability. After at least five generations, the inbred plant is considered genetically pure.

Each breeding program should include a periodic, objective evaluation of the efficiency of the breeding procedure. Evaluation criteria vary depending on the goal and objectives. Promising advanced breeding lines are thoroughly tested and compared to appropriate standards in environments representative of the commercial target area(s) for generally three or more years. Identification of individuals that are genetically superior is difficult because genotypic value can be masked by confounding plant traits or environmental factors. One method of identifying a superior plant is to observe its performance relative to other experimental plants and to one or more widely grown standard varieties. Single observations can be inconclusive, while replicated observations provide a better estimate of genetic worth.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued. Descriptions of other breeding methods that are commonly used for different traits and crops can be found in one of several reference books (e.g., Allard, 1960; Simmonds, 1979; Sneep et al., 1979; Fehr, 1987a,b).

The effectiveness of selecting for genotypes with traits of interest (e.g., increased yield, disease resistance, fatty acid profile) in a breeding program will depend upon: 1) the extent to which the variability in the traits of interest of individual plants in a population is the result of genetic factors and is thus transmitted to the progenies of the selected genotypes; and 2) how much the variability in the traits of interest among the plants is due to the environment in which the different genotypes are growing. The inheritance of traits ranges from control by one major gene whose expression is not influenced by the environment (i.e., qualitative characters) to control by many genes whose effects are greatly influenced by the environment (i.e., quantitative characters). Breeding for quantitative traits such as yield is further characterized by the fact that: 1) the differences resulting from the effect of each gene are small, making it difficult or impossible to identify them individually; 2) the number of genes contributing to a character is large, so that distinct segregation ratios are seldom if ever obtained; and 3) the effects of the genes may be expressed in different ways based on environmental variation. Therefore, the accurate identification of transgressive segregates or superior genotypes with the traits of interest is extremely difficult and its success is dependent on the plant breeder's ability to minimize the environmental variation affecting the expression of the quantitative character in the population.

The likelihood of identifying a transgressive segregant is greatly reduced as the number of traits combined into one genotype is increased. For example, if a cross is made between cultivars differing in three complex characters, such as yield, elevated β-conglycinin and at least a first agronomic trait, it is extremely difficult without molecular tools to recover simultaneously by recombination the maximum number of favorable genes for each of the three characters into one genotype. Consequently, all the breeder can generally hope for is to obtain a favorable assortment of genes for the first complex character combined with a favorable assortment of genes for the second character into one genotype in addition to a selected gene.

Backcrossing is an efficient method for transferring specific desirable traits. This can be accomplished, for example, by first crossing a superior variety inbred (A) (recurrent parent) to a donor inbred (non-recurrent parent), which carries the appropriate gene(s) for the trait in question (Fehr, 1987). The progeny of this cross are then mated back to the superior recurrent parent (A) followed by selection in the resultant progeny for the desired trait to be transferred from the non-recurrent parent. Such selection can be based on genetic assays or on the phenotype of the progeny plant. After five or more backcross generations with selection for the desired trait, the progeny are heterozygous for loci controlling the characteristic being transferred, but are like the superior parent for most or almost all other genes. The last generation of the backcross is selfed, or sibbed, to give pure breeding progeny for the gene(s) being transferred, for example, loci providing the plant with decreased seed glycinin content.

In one embodiment of the invention, the process of backcross conversion may be defined as a process including the steps of:
(a) crossing a plant of a first genotype containing one or more desirable trait, such as enhanced bioactivity for human and animal health, to a plant of a second genotype lacking said desirable trait;
(b) selecting one or more progeny plant(s) containing the desirable trait
(c) crossing the progeny plant to a plant of the second genotype; and
(d) repeating steps (b) and (c) for the purpose of transferring said desirable trait from a plant of a first genotype to a plant of a second genotype.

Introgression of a particular trait into a plant genotype is defined as the result of the process of backcross conversion. A plant genotype into which a trait has been introgressed may be referred to as a backcross converted genotype, line, inbred, or hybrid. Similarly a plant genotype lacking the desired trait may be referred to as an unconverted genotype, line, inbred, or hybrid. Backcrossing can be used with the present invention to introduce the enhanced bioactivity for human and animal health trait in accordance with the current invention into any variety by conversion of that trait.

The selection of a suitable recurrent parent is an important step for a successful backcrossing procedure. The goal of a backcross protocol is to alter or substitute a trait or characteristic in the original inbred. To accomplish this, one or more loci of the recurrent inbred is modified or substituted with the desired gene from the non-recurrent parent, while retaining essentially all of the rest of the desired genetic, and therefore the desired physiological and morphological, constitution of the original inbred. The choice of the particular non-recurrent parent will depend on the purpose of the backcross, which in the case of the present invention may be to add one or more allele(s) conferring enhanced bioactivity for human and animal health. The exact backcrossing protocol will depend on the characteristic or trait being altered to determine an appropriate testing protocol. Although backcrossing methods are simplified when the characteristic being transferred is a dominant allele, a recessive allele may also be transferred. In this instance it may be necessary to introduce a test of the progeny to determine if the desired characteristic has been successfully transferred. In the case of the present invention, one may test the β-conglycinin content of progeny lines generated during the backcrossing program, for example by SDS-PAGE/Coomassie staining.

Soybean plants (Glycine max L.) can be crossed by either natural or mechanical techniques (see, e.g., Fehr, 1980). Natural pollination occurs in soybeans either by self pollination or natural cross pollination, which typically is aided by pollinating organisms. In either natural or artificial crosses, flowering and flowering time are an important consideration. Soybean is a short-day plant, but there is considerable genetic variation for sensitivity to photoperiod (Hamner, 1969; Criswell and Hume, 1972). The critical day length for flowering ranges from about 13 h for genotypes adapted to tropical latitudes to 24 h for photoperiod-insensitive genotypes grown at higher latitudes (Shibles et al., 1975). Soybeans seem to be insensitive to day length for 9 days after emergence. Photoperiods shorter than the critical day length are required for 7 to 26 days to complete flower induction (Borthwick and Parker, 1938; Shanmugasundaram and Tsou, 1978).

Either with or without emasculation of the female flower, hand pollination can be carried out by removing the stamens and pistil with a forceps from a flower of the male parent and gently brushing the anthers against the stigma of the female flower. Access to the stamens can be achieved by removing the front sepal and keel petals, or piercing the keel with closed forceps and allowing them to open to push the petals away. Brushing the anthers on the stigma causes them to rupture, and the highest percentage of successful crosses is obtained when pollen is clearly visible on the stigma. Pollen shed can be checked by tapping the anthers before brushing the stigma. Several male flowers may have to be used to obtain suitable pollen shed when conditions are unfavorable, or the same male may be used to pollinate several flowers with good pollen shed.

Genetic male sterility is available in soybeans and may be useful to facilitate hybridization in the context of the current invention, particularly for recurrent selection programs (Brim and Stuber, 1973). The distance required for complete isolation of a crossing block is not clear; however, outcrossing is less than 0.5% when male-sterile plants are 12 m or more from a foreign pollen source (Boerma and Moradshahi, 1975). Plants on the boundaries of a crossing block probably sustain the most outcrossing with foreign pollen and can be eliminated at harvest to minimize contamination.

Once harvested, pods are typically air-dried at not more than 38° C. until the seeds contain 13% moisture or less, then the seeds are removed by hand. Seed can be stored satisfactorily at about 25° C. for up to a year if relative humidity is 50% or less. In humid climates, germination percentage declines rapidly unless the seed is dried to 7% moisture and stored in an air-tight container at room temperature. Long-term storage in any climate is best accomplished by drying seed to 7% moisture and storing it at 10° C. or less in a room maintained at 50% relative humidity or in an air-tight container.

### III. UTILIZATION OF SOYBEAN SELECTION METHOD AND BIOACTIVITY DATA

The method of grinding, defatting, digesting and centrifuging the digest to provide a soybean digest can be used in any bioactivity assay deemed of value for the purpose of selecting soybean varieties with improved bioactivity and or to standardize varieties according to a bioactivity specification. Common bioactivities include cancer cell viability and related gene expressions; bile acid binding; up regulation of mRNA for liver LDL-receptor and liver enzymes such as HMG CoA reductase and fatty acid synthase; up regulation of adiponectin secretion from adipocytes; inhibition of lipid accumulation, expression of apoA1; and reduced platelet reactivity. The method of grinding, defatting, and digesting to provide a digest can be used in any bioactivity assay deemed of value for gut health. Examples of assays related to gut physiology include changes in microflora populations and their metabolites including lipopolysaccharides; mRNA expression patterns for biochemicals such as peptide YY and proglucagon in colon-type cells.

A bioactivity specification can be established for soybean varieties to enable quality control and assurance to ensure customers to have confidence that the soybeans that they purchase will have the same bioactivity every time they get a shipment. Bioactivity specifications for raw materials can make it feasible for a food product manufacturer to develop food products that have a consistent bioactivity so that their customers can have confidence to include the bioactive foods in a diet that has consistent bioactivity benefits. It is the improved diet that involves synergistic effects of numerous components of foods and ingredients that will ultimately provide the greatest benefits to consumers. However, bioactivity specifications for the raw materials are required to develop optimized diets that can be continuously improved over time. The significance of this invention is that it allows for the use of whole soybean ingredients with bioactivity specifications in healthy food and diet creation. The whole soybean approach, with bioactivity specifications, will result greater improvements in diets than the use of foods formulated with purified components from soybeans.

A non-limiting list of bioactivities of interest includes: antioxidant activity, anti-inflammatory activity, anti-fat accumulation activity (or fat burning activity), reduced cholesterol levels, reduced joint pain, reduced itching, prebiotic, antibiotic and anti-cancer activity; including reduced cancer cell viability, increased LDL receptor activity, increased inhibition of lipid accumulation, increased adiponectin expression, reduced LPL or FAS expression, inhibition of pain, inhibition of itching, inhibition of growth of pathogens, and enhanced growth of beneficial microflora in the gut of an animal.

Regarding antioxidant activity, free radicals are a natural by-product of oxidative reactions within cells. Free radicals initiate chemical chain reactions that damage cells. Oxidative stress compromises human health and contributes to disease development. Antioxidants stop such chain reactions by scavenging free radical intermediates, and inhibit additional oxidation reactions. Antioxidants can thus prevent and repair damage caused by free radicals and can inhibit such health problems as heart disease, macular degeneration, diabetes, and cancer. Antioxidants also may help to protect the nutritional value and flavor quality of food components such as omega-3 fatty acids and conjugated linoleic acid (CLA) during food storage and as they move through digestion process in the body. Soy is a natural source of antioxidant activity. However, the antioxidant activity of compositions made from soybean products has not been well-understood. Thus, the antioxidant capacity of soy protein hydrolysates prepared from seven soybean varieties (M-A2, M-B1, M-B2, M-C1, M-C2, M-D1, and M-D2) using alcalase (SAH) or simulated gastrointestinal digestion (SGIH) were evaluated to identify compositions, and sources of compositions, that display enhanced antioxidant activity (e.g. see Example 8).

Regarding prebiotic activity, gut microflora comprise microorganisms that live in the animal digestive tract and perform a number of useful functions for their hosts. Microflora may aid in the digestion of unutilized energy substrates; stimulate cell growth; repress the growth of harmful microorganisms; train the immune system to respond only to pathogens and help to defend against some diseases. In particular, Bifidobacteria promote digestion, boost the immune system, and produce lactic and acetic acid that helps to control intestinal pH. These bacteria also inhibit the growth of Candida albicans, Escherichia coli, and other bacteria that may have more pathogenic qualities. Methods to evaluate the effect of food ingredients on the flora of the large intestine have been developed (e.g. TNO; Delft and Zeist, The Netherlands; U.S. Pat. No. 5,525,305; Schaafsma, 2005; Krul et al., 2002; Minekus et al., 1999). For instance, TNO's “micro-gut” is a multi-channel system that allows both the cultivation and stabilization of the intestinal flora. In the micro-gut cultivation system, the chemical and physical conditions of the large intestine are mimicked to provide a “natural” environment for the intestinal microbial flora. In the system, many food components, or their combinations, can be tested for their effects on intestinal flora composition and activity. The system also allows simultaneous testing of the effects of a single component on the intestinal floras of different groups such as infants, people with allergies, or elderly people. These micro-gut analyses may be utilized with a microarray technique for rapid identification and characterization of shifts in the intestinal flora (e.g. U.S. Patent Application Publication 20060246444; U.S. Patent Application Publication 20080113872). This allows for evaluation of prebiotic efficacy of tested soybean products.

A soybean plant provided by the invention may be used for any purpose deemed of value. Common uses include the preparation of food for human consumption, feed for non-human animal consumption and industrial uses. As used herein, “industrial use” or “industrial usage” refers to non-food and non-feed uses for soybeans or soy-based products.

Soybeans are commonly processed into two primary products, soybean protein (meal) and crude soybean oil. Both of these products are commonly further refined for particular uses. Refined oil products can be broken down into glycerol, fatty acids and sterols. These can be for food, feed or industrial usage. Edible food product use examples include coffee creamers, margarine, mayonnaise, pharmaceuticals, salad dressings, shortenings, bakery products, and chocolate coatings.

The hydrolysis of soy protein into short chain peptides often results in a soy protein product with bitter off-flavors associated with small hydrophobic peptides. By reducing the enzyme treatment time, the extent of hydrolysis may be limited while retaining good flavor (e.g. U.S. Patent Application Publication 2008/0096243). Enzyme treatment time can be varied to optimize the flavor and bioactivity. Hydrolysis may be effected, for instance, by use of an alkaline protease, such as, for instance, Alcalase®, (Novo Nordisk A/S, Denmark); Alkaline Protease Concentrate (Valley Research, South Bend, Ind.); or Protex™ 6L (Genencor, Palo Alto, Calif.). The final bioactivity of the protease-treated soybean ingredients after each treatment time can be tested by treatment with gastric enzymes (e.g. pepsin and pancreatin) followed by bioactivity testing.

Soy protein products (e.g., meal), can be divided into soy flour concentrates and isolates which have both food/feed and industrial use. Soy flour and grits are often used in the manufacturing of meat extenders and analogs, pet foods, baking ingredients and other food products. Food products made from soy flour and isolate include baby food, candy products, cereals, food drinks, noodles, yeast, beer, ale, etc. Soybean meal in particular is commonly used as a source of protein in livestock feeding, primarily swine and poultry. Feed uses thus include, but are not limited to, aquaculture feeds, bee feeds, calf feed replacers, fish feed, livestock feeds, poultry feeds and pet feeds, etc.

Whole soybean products can also be used as food or feed. Common food usage includes products such as the seed, bean sprouts, baked soybean, full fat soy flour and soymilk used in various products of baking, roasted soybean used as confectioneries, soy nut butter, soy coffee, soy yogurt and other soy derivatives of oriental foods. For feed usage, hulls are commonly removed from the soybean and used as feed.

Soybeans additionally have many industrial uses. One common industrial usage for soybeans is the preparation of binders that can be used to manufacture composites. For example, wood composites may be produced using modified soy protein, a mixture of hydrolyzed soy protein and PF resins, soy flour containing powder resins, and soy protein containing foamed glues. Soy-based binders have been used to manufacture common wood products such as plywood for over 70 years. Although the introduction of urea-formaldehyde and phenol-formaldehyde resins has decreased the usage of soy-based adhesives in wood products, environmental concerns and consumer preferences for adhesives made from a renewable feedstock have caused a resurgence of interest in developing new soy-based products for the wood composite industry.

Preparation of adhesives represents another common industrial usage for soybeans. Examples of soy adhesives include soy hydrolysate adhesives and soy flour adhesives. Soy hydrolysate is a colorless, aqueous solution made by reacting soy protein isolate in a 5 percent sodium hydroxide solution under heat (120° C.) and pressure (30 psig). The resulting degraded soy protein solution is basic (pH 11) and flowable (approximately 500 cps) at room temperature. Soy flour is a finely ground, defatted meal made from soybeans. Various adhesive formulations can be made from soy flour, with the first step commonly requiring dissolving the flour in a sodium hydroxide solution. The strength and other properties of the resulting formulation will vary depending on the additives in the formulation. Soy flour adhesives may also potentially be combined with other commercially available resins.

Soybean oil may find application in a number of industrial uses. Soybean oil is the most readily available and one of the lowest-cost vegetable oils in the world. Common industrial uses for soybean oil include use as components of anti-static agents, caulking compounds, disinfectants, fungicides, inks, paints, protective coatings, wallboard, anti-foam agents, alcohol, margarine, paint, ink, rubber, shortening, cosmetics, etc. Soybean oils have also for many years been a major ingredient in alkyd resins, which are dissolved in carrier solvents to make oil-based paints. The basic chemistry for converting vegetable oils into an alkyd resin under heat and pressure is well understood to those of skill in the art.

Soybean oil in its commercially available unrefined or refined, edible-grade state, is a fairly stable and slow-drying oil. Soybean oil can also be modified to enhance its reactivity under ambient conditions or, with the input of energy in various forms, to cause the oil to copolymerize or cure to a dry film. Some of these forms of modification have included epoxidation, alcoholysis or trans-esterification, direct esterification, metathesis, isomerization, monomer modification, and various forms of polymerization, including heat bodying. The reactive linoleic-acid component of soybean oil with its double bonds may be more useful than the predominant oleic- and linoleic-acid components for many industrial uses.

Solvents can also be prepared using soy-based ingredients. For example, methyl soyate, a soybean-oil based methyl ester, is gaining market acceptance as an excellent solvent replacement alternative in applications such as parts cleaning and degreasing, paint and ink removal, and oil spill remediation. It is also being marketed in numerous formulated consumer products including hand cleaners, car waxes and graffiti removers. Methyl soyate is produced by the trans-esterification of soybean oil with methanol. It is commercially available from numerous manufacturers and suppliers. As a solvent, methyl soyate has important environmental- and safety-related properties that make it attractive for industrial applications. It is lower in toxicity than most other solvents, is readily biodegradable, and has a very high flash point and a low level of volatile organic compounds (VOCs). The compatibility of methyl soyate is excellent with metals, plastics, most elastomers and other organic solvents. Current uses of methyl soyate include cleaners, paint strippers, oil spill cleanup and bioremediation, pesticide adjuvants, corrosion preventives and biodiesel fuels additives.

### IV. KITS

Any of the compositions described herein may be comprised in a kit. In a non-limiting example, a composition for the detection of a polymorphism as described herein and/or additional agents, may be comprised in a kit. The kits may thus comprise, in suitable container means, a probe or primer for detection of the polymorphism and/or an additional agent of the present invention. In specific embodiments, the kit will allow detection of at least one non-transgenic enhanced bioactivity for human and animal health trait by detection of polymorphisms in such alleles and/or otherwise in linkage disequilibrium with the allele(s). For example, the kit will allow detection of alleles associated with elevated β-conglycinin content which is associated with reducing cancer cell viability, reducing lipid accumulation increasing cholesterol metabolism or increasing adiponectin expression.

The kits may comprise a suitably aliquoted agent composition(s) of the present invention, whether labeled or unlabeled for any assay format desired to detect such alleles. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The kits of the present invention also will typically include a means for containing the detection composition and any other reagent containers in close confinement for commercial sale. Such containers may include injection or blow-molded plastic containers into which the desired vials are retained.

When the components of the kit are provided in one and/or more liquid solutions, the liquid solution may be an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means. The container means will generally include at least one vial, test tube, flask, bottle, syringe and/or other container means, into which the composition for detecting a null allele are placed, preferably, suitably allocated. The kits may also comprise a second container means for containing a sterile buffer and/or other diluent.

The kits of the present invention will also typically include a means for containing the vials in close confinement for commercial sale, such as, e.g., injection and/or blow-molded plastic containers into which the desired vials are retained. Irrespective of the number and/or type of containers, the kits of the invention may also comprise, and/or be packaged with, an instrument for assisting with the use of the detection compositions.

### V. DEFINITIONS

In the description and tables which follow, a number of terms are used. In order to provide a clear and consistent understanding of the specification and claims, the following definitions are provided:

α-subunit: As used herein, means the β-conglycinin α-subunit.

α′-subunit: As used herein, means the β-conglycinin α′-subunit.

β-subunit: As used herein, means the β-conglycinin β-subunit.

A: When used in conjunction with the word “comprising” or other open language in the claims, the words “a” and “an” denote “one or more.”

Adipocyte: A fat cell, a connective tissue cell that has differentiated and become specialized in the synthesis and storage of fat. The adipocyte is important to the body in maintaining proper energy balance, storing calories in the form of lipids, mobilizing energy sources in response to hormonal stimulation, and commanding changes by signal secretions.

Adiponectin: A protein hormone produced and secreted exclusively by adipocytes that regulates the metabolism of lipids and glucose. Adiponectin influences the body's response to insulin.

Agronomically Elite: As used herein, means a genotype that has a culmination of many distinguishable traits such as seed yield, emergence, vigor, vegetative vigor, disease resistance, seed set, standability and threshability which allows a producer to harvest a product of commercial significance.

Allele: Any of one or more alternative forms of a gene locus, all of which alleles relate to a trait or characteristic. In a diploid cell or organism, the two alleles of a given gene occupy corresponding loci on a pair of homologous chromosomes.

Backcrossing: A process in which a breeder repeatedly crosses hybrid progeny, for example a first generation hybrid (F₁), back to one of the parents of the hybrid progeny. Backcrossing can be used to introduce one or more single locus conversions from one genetic background into another.

Commercially Significant Yield: A yield of grain having commercial significance to the grower represented by an actual grain yield of at least 95% of the check lines AG2703 and DKB23-51 when grown under the same conditions.

Crossing: The mating of two parent plants.

Cross-pollination: Fertilization by the union of two gametes from different plants.

Down-regulatory mutation: For the purposes of this application a down regulatory mutation is defined as a mutation that reduces the expression levels of a protein from a given gene. Thus a down-regulatory mutation comprises null mutations.

F₁ Hybrid: The first generation progeny of the cross of two non-isogenic plants.

Genotype: The genetic constitution of a cell or organism.

Glycinin null: Mutant soybean plants with mutations conferring reduced glycinin content and increased β-conglycinin content. Plants with increased β-conglycinin contents may have non-transgenic null alleles for Gy1, Gy2, Gy3, and/or Gy4.

INDEL: Genetic mutations resulting from insertion or deletion of nucleotide sequence.

Industrial use: A non-food and non-feed use for a soybean plant. The term “soybean plant” includes plant parts and derivatives of a soybean plant.

Linkage: A phenomenon wherein alleles on the same chromosome tend to segregate together more often than expected by chance if their transmission was independent.

Marker: A readily detectable phenotype, preferably inherited in co-dominant fashion (both alleles at a locus in a diploid heterozygote are readily detectable), with no environmental variance component, i.e., heritability of 1.

Median Inhibition Concentration (IC₅₀): IC₅₀ represents the concentration of an inhibitor that is required for 50% inhibition of its target (i.e. an enzyme, cell, cell receptor or a microorganism). IC₅₀ measures the amount of a particular substance/molecule is needed to inhibit some biological process by 50%.

Non-transgenic mutation: A mutation that is naturally occurring, or induced by conventional methods (e.g. exposure of plants to radiation or mutagenic compounds), not including mutations made using recombinant DNA techniques.

Null phenotype: A null phenotype as used herein means that a given protein is not expressed at levels that can be detected. In the case of the Gy subunits, expression levels are determined by SDS-PAGE and Coomassie staining.

Phenotype: The detectable characteristics of a cell or organism, which characteristics are the manifestation of gene expression.

Protein hydrolysate: Protein split into smaller units or peptides about 1 kDa to 15 kDa in molecular weight. The term can also refer to a mixture of amino acids prepared by splitting a protein with acid, alkali or enzyme—this is a complete hydrolysate rather than a partial hydrolysate which is the use in this application. Such preparations provide the nutritive equivalent of the original material in the form of its constituent amino acids and are used for their high solubility or improved bioactivity. Protein hydrolysate is also referred to as hydrolysate.

Quantitative Trait Loci (QTL): Quantitative trait loci (QTL) refer to genetic loci that control to some degree numerically representable traits that are usually continuously distributed.

SNP: Refers to single nucleotide polymorphisms, or single nucleotide mutations when comparing two homologous sequences.

Soy Protein Hydrolysate (SPH or SH): Protein hydrolysate is derived from the treatment of an aqueous suspension of soy flour with protease(s).

Soy Alcalase Hydrolysate (SAH): The soy protein hydrolysate is produced with the addition of the enzyme Alcalase®.

Soy Gastrointestinal Hydrolysates (SGIH): The soy protein hydrolysate is produced with the addition of the enzymes pepsin and pancreatin.

Stringent Conditions: Refers to nucleic acid hybridization conditions of 5×SSC, 50% formamide and 42° C.

Substantially Equivalent: A characteristic that, when compared, does not show a statistically significant difference (e.g., p=0.05) from the mean.

Tissue Culture: A composition comprising isolated cells of the same or a different type or a collection of such cells organized into parts of a plant.

Transgene: A genetic locus comprising a sequence which has been introduced into the genome of a soybean plant by transformation.

Nutraceutical: Foods that have a medicinal effect on human health.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Preparation of Bioactive Peptides from Soybean

Soybeans are associated with numerous benefits associated with cardiovascular health, weight management and healthy, non-cancerous cells. The properties and mechanisms associated with such benefits often involve additive and synergistic effects of multiple components within the soybean. Therefore, evaluating purified components of soybeans may provide limited information on effects on human and animal health. Therefore, it is important to examine whole soybean compositions for bioactivities, recognizing that a whole soybean that has one improved bioactivity will also continue to have multiple other bioactivities that are beneficial to health.

A number of soybean components are denatured and deactivated during industrial processing. Therefore, it was found to be critical to mimic the industrial processing to determine if bioactive proteins associated with human and animal health are still active after processing. Thermal treatments can model food processing conditions. First, soybeans are ground and the fat is extracted. Soybeans may also be ground in hot water (example, 80-95° C.) to simulate an industrial process, dried and defatted. The hydrated and ground soybeans could also be modified with enzymes prior to drying and defatting to simulate an industrial product. Heat-treatments are needed to inactivate bacteria and soybean and fungal enzymes such as lipoxygenases that cause food deterioration.

Many bioactive peptides are activated during digestion. Therefore, it is importation to simulate digestion. The soybean is digested for about 3 hours with pepsin followed by about 3 hours with pancreatin to produce bioactive peptides. Pancreatin includes other enzymes such as trypsin, chymotrypsin and elastase. Bioactive peptides from soybeans are produced in the presence of other soybean components such as fiber and saponins and protease inhibitors. Digestion can be stopped or slowed by a heat treatment. A centrifugation step removes high molecular weight fibers and protein fractions that would not normally be present in vivo, e.g., at the liver. Alternatively, the centrifugation step can be omitted to study gut bioactivities such as bile acid binding or prebiotic effects on microflora populations.

When the fat is not extracted from the soybean it can be digested using lipases. Alternatively, a portion of extracted fat can be digested and added back to the meal digest to stimulate the level of fat that might reach the microflora in the colon.

Whole soybean compositions were evaluated in multiple bioactivity assays to select varieties that generally optimize bioactivities related to human and animal health. Common bioactivities include reduced cancer cell viability and related gene expressions; increased bile acid binding; up regulation of mRNA for liver LDL-receptor and down regulation of liver enzymes such as HMG CoA reductase and fatty acid synthase; up regulation of adiponectin secretion from adipocytes; increased expression of apoA1; and reduced platelet reactivity; enhancement of beneficial gut bacteria such as bifidobacteria and inhibition of pathogens.

### Example 2

### Soybean Varieties with Increased Anticancer Bioactivity

Seven soybean lines, NB1-NB7, were evaluated for protein composition and anti-cancer bioactivity. In vitro digestion of defatted soy flour of soybean line NB1-NB7 (0.4%-3.1% fat, dry basis, 46.5%-52.5% protein, dry basis) and purified glycinin and β-conglycinin was performed in a way to simulate the in vivo enzyme hydrolysis. Briefly, soy samples were suspended in water (1:20 w/v) and heated at 80° C. for 5 min to denature lipoxygenase. Then a sequential enzyme digestion was carried out with pepsin (EC 3.4.23.1, 662 units/mg; enzyme/flour, 1:20 (w/w); pH 2), and pancreatin (8×USP; enzyme/flour, 1:20 (w/w); pH 7.5) at 37° C. for 3 h each. The hydrolysis was stopped by heating at 75° C. for 20 min. The resulting hydrolysate was centrifuged at 27000×g for 15 min. The supernatant was filtered through 0.22 μm PVDF (polyvinylidene fluoride) membrane and lyophilized in a FreeZone® freeze dry system. The seven respective soy protein hydrolysates (SPH) of NB1-NB7 were named as NBH1-NBH7. All samples were stored at −80° C. until analysis.

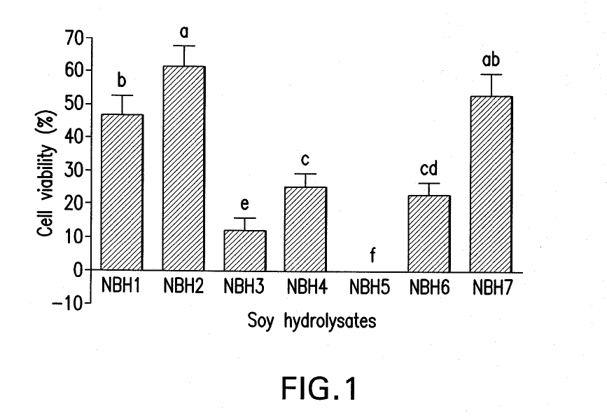

For the cytotoxicity assay, L1210 leukemia cells were cultured at 37° C. in a 5% humidified CO₂ atmosphere in Minimum Essential Medium supplemented with 10% bovine serum. The log phase cell suspension (90 μL) was plated on 96-well flat-bottom cell culture plates to make 2000 cells/well. After incubation for 24 h, the L1210 leukemia cells were treated respectively with soy protein hydrolysates (0.3-8 mg/mL), synthetic lunasin (1-80 μM), purified soy BBI (0.1-500 μM), Novasoy 400 and Novasoy 700 (total isoflavone 3-203 μM). Etoposide was included as a positive control. The plates were then incubated for 48 h at 37° C. in a 5% CO₂ atmosphere. Cell Counting Kit-8 was used to determine the number of viable cells. The results were expressed as percent viability of treated cells compared with the untreated control. FIG. 1 presents the percent L1210 leukemia cell viability after 48 h treatment with 6 mg/mL of soy protein hydrolysates. NBH5 hydrolysate showed the highest cytotoxicity to L1210 leukemia cells (100% inhibition), followed by NBH3 (87.6% inhibition), NBH6 (77.0% inhibition), NBH4 (74.5% inhibition), NBH 1 (53.1% inhibition), NBH7 (47.0% inhibition) and NBH2 (38.5% inhibition). Different hydrolysate samples showed statistically significant different activities (p<0.05), except between the pairs NBH2 & NBH7, NBH4 & NBH6, and NBH1 & NBH7. Soy protein hydrolysates inhibited L1210 leukemia cell growth in a dose dependent manner Cytotoxicity results showed that the IC₅₀ values of the hydrolysates were 5.6 (NBH1), 6.2 (NBH2), 4.6 (NBH3), 5.0 (NBH4), 3.5 (NBH5), 3.7 (NBH6) and 5.0 (NBH7) mg dry material/mL, respectively. This assay can be used to identify new germplasm with anticancer activity. It can also be used to for breeding and selection purposes.

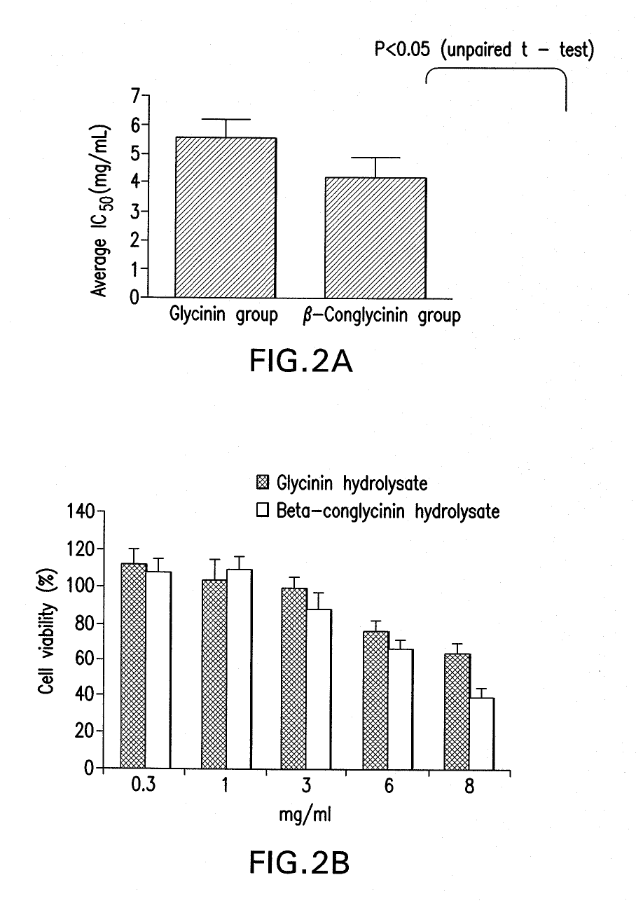

Soy hydrolysates with the highest anticancer activity were derived from soybeans with elevated β-conglycinin levels (Table 1). The total β conglycinin and total glycinin content of the seven genotypes ranged from 25.6-50.6% and 3.9-37.9%, respectively. Based on the percentage of total β-conglycinin and total glycinin, one way ANOVA analysis divided seven genotypes into two groups, a β-conglycinin featured group (NB3, NB4, NB5 and NB6, total β-conglycinin concentration >40%), and a glycinin featured group (NB1, NB2 and NB7, total glycinin concentration >30%). β-conglycinin featured group had the higher toxicity to leukemia cells (100-74.5% inhibition) compared to a glycinin featured group (53.1-38.5% inhibition). FIG. 2 presents the average IC₅₀ values of both glycinin featured group (NB1, NB2 and NB7), and the β-conglycinin featured group (NB3, NB4, NB5 and NB6). The average IC₅₀ value of the β-conglycinin featured group (4.2 mg/ml) was significantly lower than that of the glycinin featured group (5.6 mg/ml); also indicating β-conglycinin embeds more active peptides against L1210 leukemia cell growth than glycinin Higher percentage of glycinin subunits correlated with a decreased percentage of more active β-conglycinin, and therefore led to less cytotoxicity of total protein present in the hydrolysates.

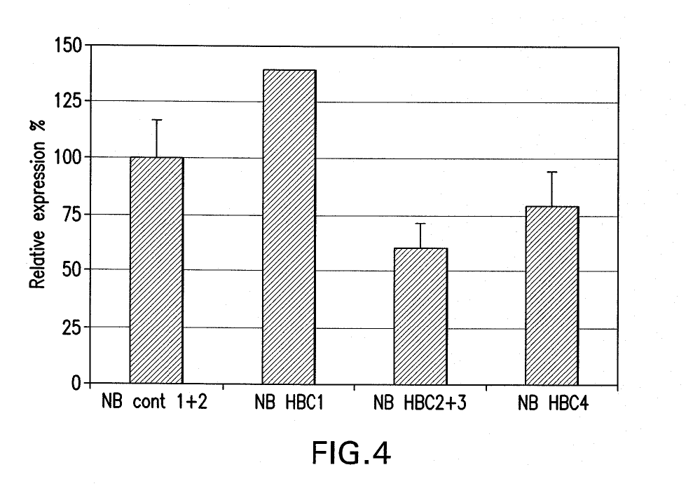

In order to confirm that β-conglycinin yields more active peptides against L1210 leukemia cell growth than glycinin, purified glycinin and β-conglycinin were hydrolyzed with pepsin and pancreatin following exactly the same procedures as for defatted soy flours. The dose dependent cytotoxicities of both hydrolysates on L1210 leukemia cells are shown in FIG. 4B. At concentration higher than 1 mg/mL, β-conglycinin hydrolysate showed significantly higher activity than glycinin hydrolysate, these results confirm the previous PLS (partial least squares) findings. The difference in the cytotoxicity of β-conglycinin and glycinin may be explained by the difference in amino acid sequence and composition. Within glycinin or β-conglycinin, different subunits share good sequence homology (Wang et al., 2005). However, between glycinin and β-conglycinin, the sequences are largely different. Unique protein sequences in β-conglycinin may embed more potent bioactive peptides. The percent acidic (Asp and Glu) and basic (Arg, His and Lys) amino acids in β-conglycinin are higher than those in glycinin. The aromatic amino acids, Phe and Tyr, are also more abundant in β-conglycinin. On the other hand, glycinin contains much higher percentage of sulfur containing amino acids Cys and Met (Mahmoud et al., 2006).

TABLE 1
Relative percentage of individual proteins as related to total protein concentrations
of NB1-NB7. The molecular weight of each protein was determined by SDS-PAGE.
The proteins were identified by comparing experimental molecular weights with
theoretical values as calculated from amino acid sequences.

| | Proteins identified | NB1 | NB2 | NB3 | NB4 | NB5 | NB6 | NB7 | LSD |
|---|---|---|---|---|---|---|---|---|---|
| Code | in soy flours | % of total protein | | | | | | | (p < 0.05) |
| P1A | lipoxygenase 2 & 3 | 5.3^{bc} | 4.9^{d} | 5.0^{cd} | 6.6^{a} | 6.9^{a} | 6.7^{a} | 5.6^{b} | 0.3 |
| P1B | lipoxygenase 1 | 1.7^{cd} | 1.6^{d} | 1.9b^{cd} | 2.0^{abc} | 2.3^{a} | 2.1^{ab} | 1.8b^{cd} | 0.4 |
| P2 | α′ subunit of β conglycinin | 8.2^{b} | 8.1^{b} | 15.5^{a} | 16.1^{a} | 16.1^{a} | 15.0^{a} | 8.3^{b} | 1.9 |
| P3 | α subunit of β conglycinin | 12.2^{b} | 12.4^{b} | 21.2^{a} | 21.4^{a} | 23.9^{a} | 21.6^{a} | 12.1^{b} | 3.0 |
| P5 | β subunit of β conglycinin | 5.2^{c} | 5.5^{c} | 12.1^{ab} | 12.3^{ab} | 10.5^{b} | 13.2^{a} | 6.0^{c} | 2.0 |
| P6 | glycinin A3 chain | 2.3^{a} | 2.5^{a} | 0.0^{b} | 0.0^{b} | 0.0^{b} | 0.0^{b} | 2.7^{a} | 0.5 |
| P7 | glycinin A1,2,4 chains | 15.6^{a} | 15.6^{a} | 3.4^{b} | 1.5^{b} | 1.7^{b} | 3.4^{b} | 14.4^{a} | 2.8 |
| P11 | Glycinin basic chains | 19.8^{a} | 19.8^{a} | 4.5^{b} | 2.3^{b} | 2.3^{b} | 4.7^{b} | 17.6^{a} | 2.7 |
| P16 | Kunitz trypsin inhibitor | 3.5^{b} | 3.4^{b} | 4.8^{a} | 4.9^{a} | 4.6^{a} | 1.5^{c} | 3.5^{b} | 1.0 |
| | Total β-conglycinin | 25.6^{b} | 25.9^{b} | 48.9^{a} | 49.8^{a} | 50.6^{a} | 49.9^{a} | 26.4^{b} | 6.4 |
| | Total glycinin | 37.7^{a} | 37.9^{a} | 7.9^{b} | 3.9^{b} | 4.0^{b} | 8.1^{b} | 34.7^{a} | 5.7 |

¹Means with different superscript letters in the same row are significantly different (p < 0.05).

The composition of elevated β-conglycinin levels may be used to identify and breed other potential soybean lines with anticancer activity.

After an appropriate phenotyping assay is determined, molecular markers are developed to assist with introgression and selection of new varieties with anti-cancer traits. Molecular markers associated with anticancer activities are obtained by a number of methods. It may be carried out by first preparing an F₂ population by selfing an F₁ hybrid produced by crossing inbred varieties only one of which comprises anticancer phenotype. Recombinant inbred lines (RIL) (genetically related lines; usually >F₅, developed from continuously selfing F₂ lines towards homozygosity) are prepared and used as a mapping population. Information obtained from dominant markers is maximized by using RIL because all loci are homozygous or nearly so.

Backcross populations (e.g., generated from a cross between a desirable variety (recurrent parent) and another variety (donor parent)) carrying a trait not present in the former are also be utilized as a mapping population. A series of backcrosses to the recurrent parent are made to recover most of its desirable traits. Thus a population is created consisting of individuals similar to the recurrent parent but each individual carries varying amounts of genomic regions from the donor parent. Backcross populations are useful for mapping dominant markers if all loci in the recurrent parent are homozygous and the donor and recurrent parent have contrasting polymorphic marker alleles (Reiter et al., 1992).

Useful populations for mapping purposes are near-isogenic lines (NIL). NILs are created by many backcrosses to produce an array of individuals that are nearly identical in genetic composition except for the desired trait or genomic region are used as a mapping population. In mapping with NILs, only a portion of the polymorphic loci are expected to map to a selected region.

### Example 3

### Soybean Varieties with Improved Bioactivity Related to Cholesterol Metabolism by the Liver

Soybean breeding lines have been developed with different functional protein composition. The LDL receptor activation of functional peptides that are produced from soybean compositions during/after passage through the gastrointestinal tract, using an in vitro model which simulates human digestion was evaluated. The conditions around oral ingested compounds (e.g. foods, ingredients) in the gastrointestinal tract are changing in time, due to the dynamic and successive conditions in the stomach and (small) intestine, whether or not as physiological reaction on the intake of the product. Accurate simulations of the in vivo conditions were performed in dynamic gastrointestinal models. The ground soybean was digested in the gastrointestinal model and exposed to cell line Hep G2 (hepablastoma derived cell line that mimics normal hepatocytes). Hep G2 was derived from Hans Princen (TNO Laboratory, Leiden, The Netherlands). Cells were cultured in DMEM with 4.5 g/l glucose, L-glutamine and without pyruvate with 10% FCS and penicillin/streptomycin. For the incubation with the digested soybean samples, the cells were cultured in 12 wells plates until confluency. Samples were diluted 1:1 with culture medium with 0.5% human serum albumin (HSA) instead of 10% FCS were incubated for 20 hours.

Following incubation, cells were carefully washed with PBS (2 times). Total RNA was isolated and reverse transcribed using RevertAid™ reverse transcriptase. Gene expression analysis was performed using real-time PCR with the primers displayed in Table 2. Hypoxanthine guanine phosphoribosyl transferase (HPRT) and 18S rRNA were used as the standard housekeeping genes. Relative gene expression (RE) numbers were calculated by subtracting the threshold cycle number (Ct) of the target gene from the average Ct of HPRT and 18S rRNA (Ct housekeeping) and raising 2 to the power of this difference. The average Ct of two housekeeping genes was used to exclude that changes in the relative expression were caused by variations in the separate housekeeping gene expressions.

TABLE 2
Primers for quantitative real-time
PCR analysis (SEQ ID NOs: 1-8).

| | Forward | Reverse |
|---|---|---|
| Gene | Primer | Primer |
| Human-LDLR | ACCACGGTGGA | GGCTTCTTCTC |
| | GATAGTGACAA | ATTTCCTCTGC |
| Human-HMGCR | TGGCTGGGAG | CAGGCAATGT |
| | CATAGGAGG | AGATGGCGGT |
| Human-18S-rRNA | CCATTCGAA | GTCACCCGTG |
| | CGTCTGCCC | GTCACCATG |
| Human-HPRT | GAAATGTCAGTT | ACAATCCGCC |
| | GCTGCATTCCT | CAAAGGGAAC |

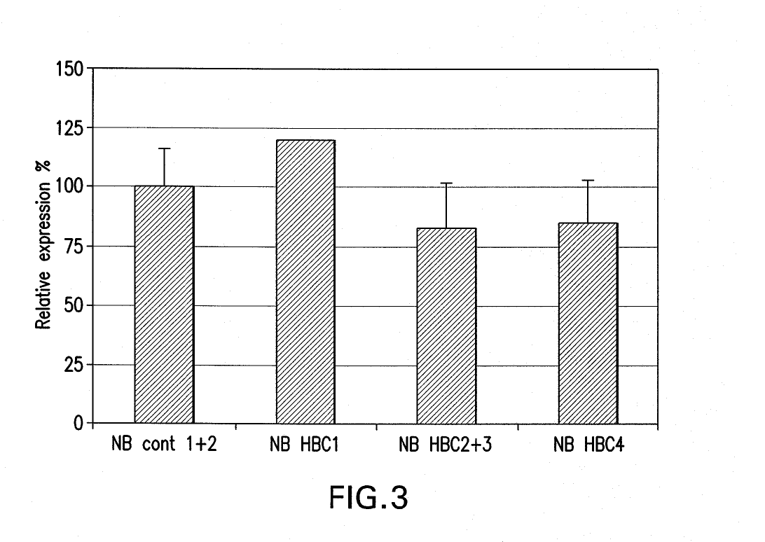

When compared to the two control soy samples (NB-cont1 and NB-cont2), NB-HBC1 had an enhancing effect on the mRNA expression of the LDL receptor (1.2 fold). For the HMG-CoA reductase mRNA NB-HBC-1 had an enhancing effect (1.4 times) (FIGS. 3-4). The enhancing effect of the LDL receptor would have a positive effect on lowering the plasma cholesterol levels in vivo. The improved bioactivity of NB-HBC-1 relative to the other HBC lines may be related to the low levels of KTI (Kunitz trypsin inhibitor) in this line (NB-HBC-1 is the same variety as NB-6 in Example 4). This assay can be used to identify new germplasm with increased LDL receptor activity. It can also be used to phenotype a mapping population for the development of molecular markers for breeding and selection purposes.

### Example 4

Soybean Varieties with Improved Bioactivity Related to Inhibiting Lipid Accumulation

Obesity is an increasing health concern in developed nations; it is a well recognized predictor of premature mortality. A number of studies suggest that consumption of soy protein has favorable effects on obesity by suppression of food intake and increased satiety and/or energy expenditure that may reduce body fat and weight. Soybean varieties were selected that have improved potential to inhibit fat accumulation in adipocytes by testing the effects of soy hydrolysates prepared from soybeans having a range of protein subunit compositions on inhibition of lipid accumulation. Fifteen soy genotypes exhibiting different protein composition profiles were evaluated for anti-adipogenesis potential (Tables 3-4). One chymotrypsin inhibition unit (CTIU) is defined as a decrease of 0.01 absorbance unit at 275 nm in a 1 cm path length cell per 10 mL of final reaction volume after 10 min. of reaction. CTIU were determined as per Kakade et al., (1970).

TABLE 3
Compositions of soybean genotypes evaluated for anti-adipogenesis potential.

| | Soybean Line | | | | | | | |
|---|---|---|---|---|---|---|---|---|
| | NB2 | NB3 | NB4 | NB5 | NB6 | NB7 | M-A1 | M-A2 |
| Compound | % Total proteins | | | | | | | |
| Lipoxygenase 2 & 3 | 4.9^{ef} | 5.3^{ed} | 6.3^{bc} | 6.9^{ab} | 7.0^{ab} | 5.9^{cd} | 4.0^{g} | 5.1^{e} |
| Lipoxygenase 1 | 1.7^{cd} | 2.0^{b} | 1.9^{b} | 2.4^{a} | 2.0^{b} | 1.8^{bc} | 1.2^{e} | 1.6^{d} |
| α′ subunit of β- | 8.7^{f} | 15.1^{cde} | 16.1^{bc} | 15.1^{cde} | 15.7^{cd} | 8.3^{f} | 8.2^{f} | 8.1^{f} |
| conglycinin | | | | | | | | |
| α subunit of β- | 11.9^{efg} | 20.4^{abc} | 20.8^{ab} | 20.8^{ab} | 22.2^{a} | 11.6^{fg} | 11.1^{fg} | 10.4^{g} |
| conglycinin | | | | | | | | |
| β subunit of β- | 5.6^{ef} | 12.4^{a} | 10.4^{b} | 10.5^{d} | 12.9^{a} | 6.3^{e} | 5.2^{fg} | 4.9^{fg} |
| conglycinin | | | | | | | | |
| Glycinin A3 chain | 2.6^{c} | 0.0^{d} | 0.0^{d} | 0.0^{d} | 0.0^{d} | 2.9^{bc} | 3.1^{b} | 3.1^{bc} |
| Glycinin A1,2,4 chains | 15.9^{a} | 3.8^{c} | 1.8^{e} | 2.0^{e} | 2.6^{ed} | 14.9^{b} | 15.3^{ab} | 14.6^{b} |
| Glycinin basic chains | 17.1^{a} | 3.9^{c} | 2.7^{c} | 2.4^{c} | 3.3^{c} | 16.7^{a} | 17.2^{a} | 17.5^{a} |
| Kunitz trypsin inhibitor | 3.7^{ed} | 4.7^{cd} | 5.2^{bc} | 4.8^{bcd} | 1.5^{f} | 3.3^{e} | 5.5^{bc} | 5.1^{bc} |
| Total b-conglycinin | 26.2^{d} | 48.0^{ab} | 47.2^{b} | 46.5^{b} | 50.8^{a} | 26.2^{d} | 24.5^{d} | 23.4^{d} |
| Total Glycinin | 35.6^{a} | 7.8^{c} | 4.5^{d} | 4.4^{d} | 5.9^{cd} | 34.5^{a} | 35.7^{a} | 35.3^{a} |

mg/g flour

| | | | | | | | | |
|---|---|---|---|---|---|---|---|---|
| Total isoflavones | 1.66^{f} | 2.07^{f} | 2.11^{f} | 6.62^{a} | 5.00^{cd} | 4.79^{d} | 3.46^{e} | 3.52^{e} |
| Total saponins | 4.66 | 3.25 | 3.16 | 1.2 | 0.8 | 1.66 | 7.87 | 7.86 |

CTIU (dry basis)

| | | | | | | | | |
|---|---|---|---|---|---|---|---|---|
| Chymotrypsin | 11 | 20 | 20 | 25 | 20 | 14 | 17.5 | 13.6 |
| inhibitor | | | | | | | | |

*Means with different superscript letters in the same row are significantly different (p < 0.0001)

TABLE 4
Additional composition information of soybean genotypes evaluated for
anti-adipogenesis potential.

| | Soybean line | | | | | | |
|---|---|---|---|---|---|---|---|
| | M-A3 | M-B1 | M-B2 | M-C1 | M-C2 | M-D1 | M-D2 |
| Compound | % Total proteins | | | | | | |
| Lipoxygenase 2 & 3 | 5.0^{e} | 3.2^{h} | 5.2^{de} | 5.5^{de} | 4.2^{fg} | 7.2^{a} | 6.9^{ab} |
| Lipoxygenase 1 | 1.3^{e} | 0.0^{f} | 0.0^{f} | 2.3^{a} | 1.6^{cd} | 1.9^{b} | 1.8^{bc} |
| α′ subunit of β- | 8.2^{f} | 14.1^{de} | 13.5^{e} | 18.0^{b} | 17.7^{b} | 20.7^{a} | 20.1^{a} |
| conglycinin | | | | | | | |
| α subunit of β- | 10.4^{g} | 18.6^{dc} | 17.7^{d} | 20.8^{ab} | 20.0^{bc} | 13.7^{e} | 13.1^{ef} |
| conglycinin | | | | | | | |
| β subunit of β- | 4.5^{g} | 8.3^{cd} | 11.3^{b} | 7.7^{d} | 9.2^{c} | 8.6^{cd} | 7.6^{d} |
| conglycinin | | | | | | | |
| Glycinin A3 chain | 2.9^{bc} | 5.6^{a} | 5.5^{a} | 0.0^{d} | 0.0^{d} | 0.0^{d} | 0.0^{d} |
| Glycinin A1,2,4 chains | 14.8^{b} | 2.1^{e} | 1.8^{e} | 2.6^{d} | 3.0^{d} | 0.0^{f} | 0.0^{f} |
| Glycinin basic chains | 17.7^{a} | 7.0^{b} | 6.5^{b} | 3.5^{c} | 3.0^{c} | 0.0^{d} | 0.0^{d} |
| Kunitz trypsin inhibitor | 5.1^{bc} | 7.1^{a} | 5.9^{b} | 5.9^{b} | 5.6^{bc} | 7.5^{a} | 7.5^{a} |
| Total b-conglycinin | 23.1^{d} | 40.9^{c} | 42.5^{c} | 46.5^{b} | 46.9^{b} | 43.0^{c} | 40.8^{c} |
| Total Glycinin | 35.4^{a} | 14.6^{b} | 13.7^{b} | 6.1^{cd} | 6.0^{cd} | 0.0^{e} | 0.0^{e} |

mg/g flour

| | | | | | | | |
|---|---|---|---|---|---|---|---|
| Total isoflavones | 3.89^{e} | 5.36^{c} | 6.37^{ab} | 6.28^{ab} | 6.05^{b} | 6.01^{b} | 5.35^{c} |
| Total saponins | 9.27 | 9.24 | 8.85 | 7.02 | 8.25 | 9.45 | 9.9 |

CTIU (dry basis)

| | | | | | | | |
|---|---|---|---|---|---|---|---|
| Chymotrypsin | 14.8 | 23.4 | 16.9 | 20.2 | 21.1 | 27.9 | 29.1 |
| inhibitor | | | | | | | |

*Means with different superscript letters in the same row are significantly different (p < 0.0001)

Cell cultures were prepared to evaluate anti-adipogenesis potential of the 15 soybean hydrolysates described in Table 3-4. 3T3-L1 (also designated as ATCC® CCL-92.1™) fibroblasts from Swiss albino mouse were obtained from the American Type Culture Collection (Manassas, Va.). The 3T3-L1 preadipocytes were seeded at 6×10³ cells/cm² in 6-well plates and cultured in DMEM containing 10 mM sodium pyruvate, 100 U/mL penicillin, 100 U/mL streptomycin and 10% FBS (FBS/DMEM medium). For induction of preadipocytes differentiation, two days after reaching 100% confluence, the cells were stimulated with FBS/DMEM medium containing 167 nM insulin, 0.5 M IBMX, and 1 M DEX for 2 days. Cells were then maintained in FBS/DMEM medium with 167 nM insulin for another 2 days, followed by culturing with FBS/DMEM medium for an additional 4 days, at which time up to 90% of cells were mature adipocytes with accumulated fat droplets. The mature adipocytes were used to evaluate the soy hydrolysates of the 15 genotypes described in Tables 3-4.

To produce the soy protein alcalase hydrolysates (SAH), soybeans were ground and defatted (2.9%-7.0% fat, dry basis; 45.3%-50.6% fat, dry basis). Next, 2 g of defatted flour, non-fat dry milk, purified β-conglycinin or glycinin were added to 25 mL of deionized water and brought to 50° C. at pH 8.0. Then, 5 mg of Alcalase® (Novo Nordisk; 11 U/mg) were added. Hydrolysis was allowed to run for 3 h, and temperature and pH were stabilized using 0.5 M NaOH at 50° C. Hydrolysis was stopped by the addition of 75 μL of 0.1 N HCl. Hydrolysates were centrifuged at 14,000×g at 10° C. for 30 min. After centrifugation, 10% trichloroacetic acid (TCA) was added in a 1:1 ratio. Once TCA was added, the hydrolysates were centrifuged again under the same conditions, and the liquid hydrolysates were filtered using stirred ultra-filtration cell 1 kDa membrane. The peptides were freeze dried in a FreeZone freeze dry system and kept at −80° C. Bradford protein assay was followed for protein quantification using a standards curve using bovine serum albumin (BSA) (y=0.0002x−0.0021, R²=0.997). Alcalase hydrolysates were further hydrolyzed with pepsin-pancreatin to mimic gastrointestinal digestion following the method described by Wang et al. (2008). Table 5 provides the names of the hydrolysates derived from the 15 soybean genotypes described in Tables 3-4.

TABLE 5
Nomenclature for alcalase hydrolysates derived from 15 soybean
genotypes.

| | | Alcalase |
|---|---|---|
| Soybean | Alternative Name | Hydrolysates |
| NB-2 | | SH1 |
| NB-3 | | SH2 |
| NB-4 | | SH3 |
| NB-5 | | SH4 |
| NB-6 | | SH5 |
| NB-7 | | SH6 |
| M-A1 | A1 | SH7 |
| M-A2 | A2 | SH8 |
| M-A3 | A3 | SH9 |
| M-B1 | B1 | SH10 |
| M-B2 | B2 | SH11 |
| M-C1 | C1 | SH12 |
| M-C2 | C2 | SH13 |
| M-D1 | D1 | SH14 |
| M-D2 | D2 | SH15 |

Mature adipocytes were treated with soy hydrolysates derived from the 15 genotypes, purified β-conglycinin hydrolysates, and glycinin alcalase hydrolysates dissolved in water at a concentration of 100 μM and incubated at 37° C. in a 5% CO₂ atmosphere for 72 h for determination of lipid accumulation. Lipid accumulation was quantified using the Oil Red O Assay. Treated adipocytes were washed with Dulbecco's Phosphate Buffered Saline (DPBS) and fixed with 10% formalin (in DPBS) in 6-well plates for 1 h. Then, cells were washed with 60% isopropanol and let air dried. The Oil Red O stock solution (6:4 v/v with water) was added to lipid droplets for 10 min. After Oil Red O lipid staining, cells were washed with water four times and were air dried. Oil Red O dye was eluted by adding 100% isopropanol after 10 min incubation at room temperature. OD₅₁₀ₙₘ of eluted isopropanol was measured using a CytoFluor Series 4000® multi-well luminescence plate reader. Inhibition percentages of lipid accumulation were calculated using the following equation:

(A_{control, 510 nm}A_{treatment, 510 nm})/A_{control, 510 nm}*100=% inhibition of lipid content

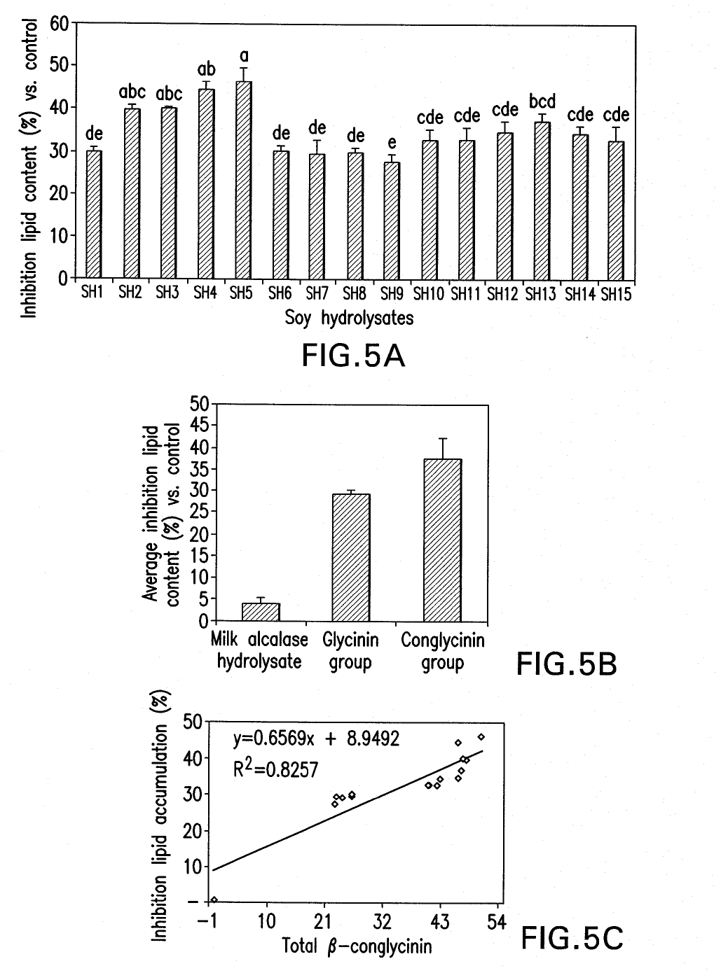

Treatment with soy alcalase hydrolysates at a concentration of 100 μM decreased lipid accumulation in adipocytes compared to untreated cells (FIG. 5A). SH2-SH5, which have 43.5%-50.8% β-conglycinin in the defatted flour, inhibited lipid accumulation by 39-46% (FIG. 5a.). SH10-SH15, which have 40.8%-46.9% β-conglycinin in the defatted flour, inhibited lipid accumulation by 33-37%. SH1, SH6, SH7, SH8 and SH9, which have 23.4%-26.7% β-conglycinin in the defatted flour, exhibited the lowest reduction of lipid content ranging from 27 to 30%.

The 15 genotypes were clustered into two groups, high β-conglycinin (SH2-SH5 and SH10-SH15) and low β-conglycinin (SH1 and SH6-SH9), for further data analysis. FIG. 5B presents the average inhibition of lipid content of both the low β-conglycinin (SH1 and SH6-SH9) and high β-conglycinin group (SH2-SH5 and SH10-SH15). The average inhibition of lipid content of the high β-conglycinin group (37.5%) was significantly higher than that of the low β-conglycinin group (29.3%), suggesting that β-conglycinin embeds more active peptides with an effect on adipocyte lipid accumulation. Furthermore, higher total β-conglycinin soy varieties correlated (R²=0.83) with higher percentage of inhibition of lipid accumulation (FIG. 5C).

Milk is widely consumed high quality protein source. Therefore, soy hydrolysates and milk alcalase hydrolysate were compared for inhibitory effect of lipid accumulation (FIG. 5C). Milk alcalase hydrolysate inhibited lipid accumulation by 4% compared with soy hydrolysate that inhibited lipid accumulation by 27%-46%. Therefore, soy hydrolysates, especially with high levels of β-conglycinin, may be useful as a significant component of weight management programs.

The relationship between bioactive soy components (X variables, including matrix protein profile, concentrations of total isoflavones, total saponins in soy hydrolysates) and either the inhibition (%) of lipid accumulation or adiponectin expression values of the fifteen soy hydrolysates (SH1-SH15) on 3T3-L1 adipocytes (Y variable) was evaluated using a partial least squares analysis. Unlike linear regression, partial least squares analysis does not give a simple regression formula. The regression model can be built, and indicates the importance of each soy component on inhibition of lipid accumulation and total adiponectin induction. To obtain an overall statistical evaluation of the contribution of each variable to the final activity, regression coefficients (B) were calculated from weights of the first (protein content) and second variable. The higher the absolute B value the higher the impact on inhibition of lipid accumulation or total adiponectin induction. The sign indicates a positive (“+” sign) or negative (“−” sign) effect on inhibition percentage of lipid accumulation or total fold-increase of adiponectin vs. control values. Variable importance for projection (VIP) of Wold (Wold, 1985) was also calculated to summarize each variables contribution to fit of the PLS model for both variables and response. In this study, a VIP value of <0.9 was considered to be small, indicating the variable was not important to the model.

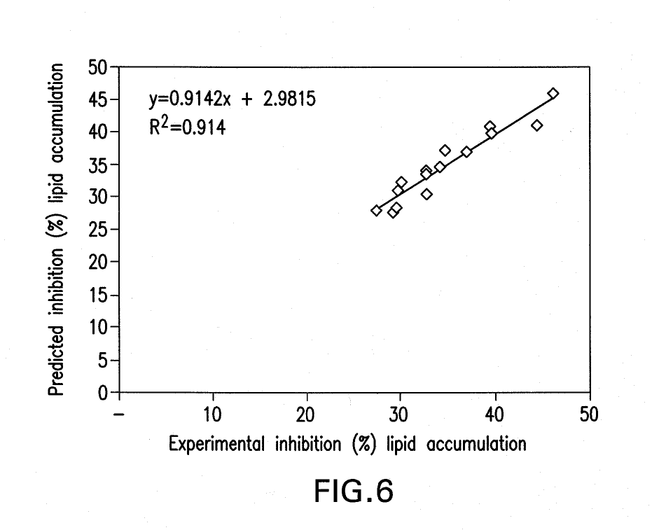

The effects of the individual protein components on lipid accumulations in adipocytes were evaluated using the partial least squares analysis. Table 6 presents the B and VIP values for each variable, arranged by positive or negative B values and then sorted by VIP values. All β-conglycinin subunits α (B=0.30, VIP=1.29), β (B=0.29, VIP=1.29), and α′ (B=0.09, VIP=0.94) showed a VIP of >0.9 and a positive regression coefficient, indicating a positive correlation with inhibition of lipid accumulation and concentration of the subunit. Conversely, all of the major glycinin chains, A3 chain (B=−0.12, VIP=0.90), basic chains (B=−0.12, VIP=1.07), A1, 2 & 4 chains (B=−0.11, VIP=0.99) and Kunitz trypsin inhibitor (B=−0.29, VIP=0.92) showed a large VIP value and a negative regression coefficient suggesting a negative correlation with inhibition of lipid accumulation and concentration of the chain. FIG. 6 is an example showing a good correlation between the predicted lipid accumulation inhibition values calculated by the partial least squares regression model and the experimental values (R²=0.91).

TABLE 6
Estimated partial least squares (PLS) weight of first (inhibition of lipid
accumulation) and second variables (protein content). Regression
coefficients (B) and variable importance to projection (VIP).

| Variables | PLS regression coefficients (B) | VIP |
|---|---|---|
| α subunit of β-conglycinin | 0.30 | 1.29 |
| β subunit of \tilde{β} conglycinin | 0.29 | 1.29 |
| α′ subunit of β-conglycinin | 0.09 | 0.94 |
| Lipoxygenase 1 | 0.09 | 0.74 |
| Lipoxygenase 2&3 | 0.11 | 0.63 |
| Kunitz trypsin inhibitor | −0.29 | 0.92 |
| Glycinin A3 chain | −0.12 | 0.90 |
| Glycinin basic chains | −0.11 | 1.07 |
| Glycinin A1, 2, 4 chains | −0.10 | 0.99 |

^{1.} Regression coefficients (B) were calculated to obtain an overall statistical evaluation of the contribution of each variable to the final activity. The higher the absolute B value is, the higher the impact, while the sign indicates positive (“+” sign) or negative (“−” sign) effect on inhibition of lipid accumulation value. Variable Importance for Projection (VIP) of Wold, was calculated to summarize each variable contribution to fit the PLS model for both variables and response. A VIP value less than 0.9 (Wold, 1995) was considered small, indicating the variable was not important to the model.

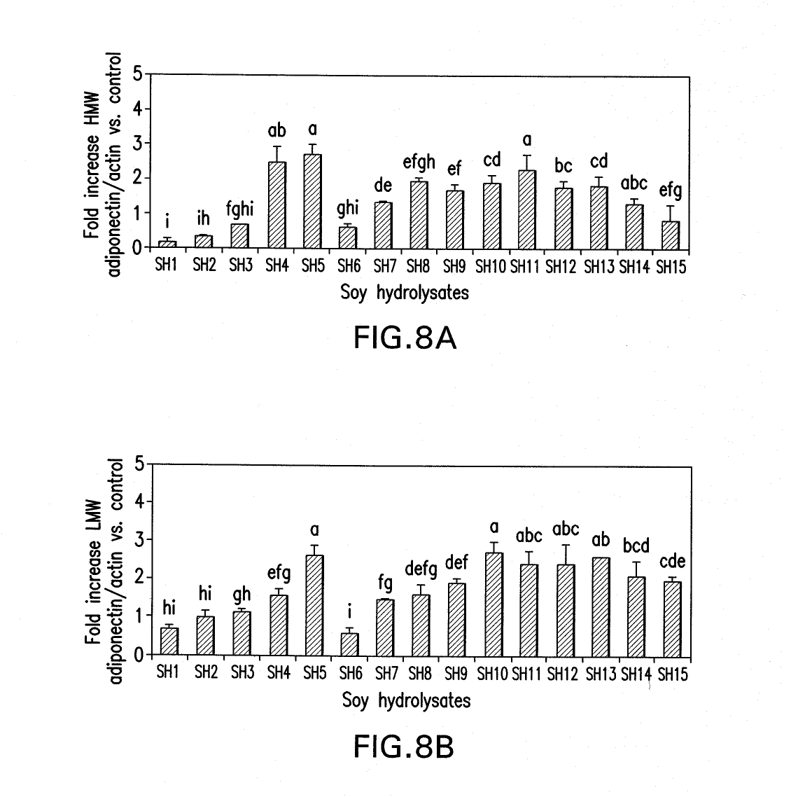

The partial least squares regression study suggested that the matrix protein composition was important to potential anti-obesity activity. β-conglycinin embedded more active peptides that induced inhibition of lipid accumulation and adipocytes than glycinin. To confirm the contributions of β-conglycinin and glycinin hydrolysates, adipocytes were treated with purified samples of β-conglycinin and glycinin hydrolysates and lipid accumulation was evaluated. FIG. 8 shows a dose-dependent inhibition of lipid accumulation vs. control in 3T3-L1 adipocytes after 72 h exposure to pure β-conglycinin and glycinin alcalase hydrolysates. At concentrations 10 and 100 μM β-conglycinin hydrolysates showed significantly higher inhibition of lipid accumulation (5.1 and 44.8% inhibition, respectively) than glycinin hydrolysates (−8.0 and −18.5% inhibition, respectively); these results confirm the previous partial least squares findings, suggesting β-conglycinin is an important component in reducing lipid accumulation.

### Example 5

### Soybean Varieties with Improved Bioactivity Related to Stimulating Adiponectin Expression

Insulin resistance and excessive body fat are both associated with lower plasma adiponectin concentrations. Therefore, soybean protein could have further value if its intake stimulates adiponectin levels in plasma. Mature adipocytes were treated with soy hydrolysates from the 15 genotypes described in Tables 3-4, purified β-conglycinin hydrolysates, and glycinin alcalase hydrolysates dissolved in water at a concentration of 100 μM and incubated at 37° C. in a 5% CO₂ atmosphere for 24 h for detection of adiponectin expression. Adiponectin expression was evaluated using Western blotting. High molecular weight adiponectin induction was detected in 3T3-L1 adipocytes treated with soy hydrolysates SH4, SH5 and SH7-SH14 at a concentration of 100 μM. SH4, SH5 and SH11 showed the highest induction of adiponectin (p<0.0001) (2.49, 2.71 and 2.27 fold increase, respectively) compared to the control (FIG. 8A). Similarly, low molecular weight adiponectin was induced when cells were treated with soy hydrolysates SH3-SH5 and SH7-SH15 at a concentration of 100 μM. SH5 and SH10-SH13 exhibited significantly (p<0.0001) higher induction of low molecular weight adiponectin levels (2.63, 2.73, 2.43, 2.43 and 2.57 fold increase, respectively) compared to the control (FIG. 8B).

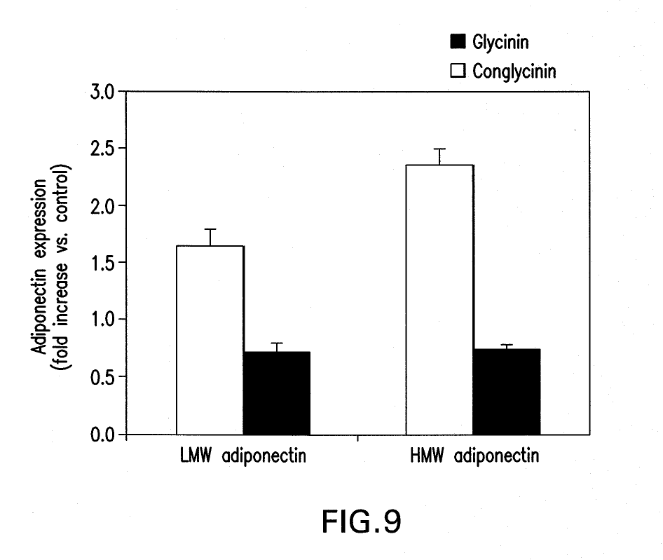

The effects of the individual protein components on of the adiponectin expression in adipocytes were evaluated using the partial least squares analysis described in example 4. Table 7 presents the B and VIP values for each variable, arranged by positive or negative B values and then sorted by VIP values. All β-conglycinin subunits α(B=0.15, VIP=1.18), α′ (B=0.09, VIP=1.01) and β (B=0.07, VIP=0.92) showed a VIP of >0.9 and a positive regression coefficient, suggesting a positive correlation with total adiponectin induction and concentration of the subunit. In contrast, lipoxygenase 2 & 3 (B=−0.21, VIP=1.23) and all of the major glycinin chains such as A1, 2 & 4 chains (B=−0.16, VIP=1.35) and basic chains (B=−0.09, VIP=1.08) showed a VIP of >0.9 and a negative regression coefficient, suggesting a negative correlation with total adiponectin induction and concentration of the subunit. Moreover, a predicted value can be calculated from partial least squares model. The partial least squares regression study suggested that the matrix protein composition was important to potential anti-obesity activity. β-conglycinin embedded more active peptides that induced total adiponectin expression in adipocytes than glycinin. To confirm the contributions of β-conglycinin and glycinin hydrolysates, adipocytes were treated with purified samples of β-conglycinin and glycinin hydrolysates and adiponectin expression was evaluated. FIG. 9 shows adiponectin induction (fold increase vs. control) in adipocytes after 24 h exposure to 100 μM pure β-conglycinin and glycinin alcalase hydrolysates. The results showed that adipocytes treated with β-conglycinin hydrolysates exhibited higher low molecular weight and high molecular weight adiponectin expression (1.64 and 2.36 fold increase vs. control, respectively) compared to glycinin alcalase hydrolysates (0.72 and 0.74 fold increase vs. control, respectively).

TABLE 7
Estimated partial least squares (PLS) weight of first (total adiponectin
induction) and second variables (protein content); Regression coefficients
(B) and variable importance to projection (VIP).

| Variables | PLS regression coefficients (B) | VIP |
|---|---|---|
| α subunit of β-conglycinin | 0.15 | 1.18 |
| α′ subunit of β-conglycinin | 0.09 | 1.01 |
| β subunit of β-conglycinin | 0.07 | 0.92 |
| Kunitz trypsin inhibitor | 0.04 | 0.44 |
| Glycinin A3 chain | 0.13 | 0.87 |
| Lipoxygenase 2&3 | −0.21 | 1.23 |
| Glycinin A1, 2, 4 chains | −0.16 | 1.35 |
| Glycinin basic chains | −0.09 | 1.08 |
| Lipoxygenase 1 | −0.04 | 0.51 |

^{1.} Regression coefficients (B) were calculated to obtain an overall statistical evaluation of the contribution of each variable to the final activity. The higher the absolute B value is, the higher the impact, while the sign indicates positive (“+” sign) or negative (“−” sign) effect on total adiponectin induction value. Variable Importance for Projection (VIP) of Wold, was calculated to summarize each variable contribution to fit the PLS model for both variables and response. A VIP value less than 0.9 (Wold, 1995) was considered small, indicating the variable was not important to the model.

### Example 6

### Soybean Varieties with Improved Bioactivity Related to Inhibiting Lipid Accumulation and Anti-Inflammatory Effects

Eight soybean genotypes (A1-D2) with varying protein profiles were ground and defatted. Next, soybean hydrolysates, either soybean gastrointestinal hydrolysate (SGIH) or soybean alcalase hydrolysates (SAH), were produced from the soybeans as described in Example 2 or Example 4, respectively. Mature adipocytes were prepared from 3T3-L1 preadipocytes. The mature adipocytes were treated separately with soy hydrolysates dissolved in water at a concentration of 100 μM and incubated at 37° C. in a 5% CO₂ atmosphere for 72 h. Gastrointestinal (GI) digestion may modify activity of bioactive peptides of SAH as a consequence of continued proteolytic hydrolysis by GI enzymes. Therefore, lipid accumulation was evaluated after simulated GI digestions of SAH. Lipids were quantified in the adipocytes by the Oil Red O assay (e.g. Janderova et al., 2003). Table 8 presents the percentage of inhibition of lipid accumulation in 3T3-L1 adipocytes after 72 h of treatment with 100 μM SAH derived from a given genotype (A1-D2) compared to their controls. Treatment with 100 μM SAH decreased lipid accumulation in 3T3-L1 adipocytes from 29% to 37% compared to negative control (untreated cells). Furthermore, SAH derived from soybean genotypes with an average of 45.5% β-conglycinin (C1, C2, D1) showed a significant higher inhibitory effect compared to SAH derived from 24% BC (A1 and A2) (P<0.05). To confirm this finding a linear correlation analysis was performed between total BC concentration and inhibition on lipid accumulation. Higher total β-conglycinin soy varieties correlated (R²=0.85) with higher percentage of inhibition on lipid accumulation (Table 8). SGIH (100 μM) inhibited lipid accumulation from 7.6% to 14.7% in 3T3-L1 adipocytes after 72 h compared to negative control (Table 8). SGIH made from the soybean type with an average of 20.4% of the protein as the α subunit of β-conglycinin (M-C1, M-C2) significantly improved the inhibition of lipid accumulation compared to soybeans with an average of 10.8% of the protein as the α subunit (M-A1, M-A2).

TABLE 8
Inhibitory effect of SAH (100 μM) and SGIH (100 μM) on lipid
accumulation relative to negative control (untreated cells) in 3T3-L1
adipocytes after 72 h.

| | β-conglycinin* | Lipid accumulation | |
|---|---|---|---|
| Soybean | (% total | (% inhibition)* | |
| genotype | protein) | SAH | SGIH |
| M-A1 | 24.5^{c} | 29.17^{c} | 7.57^{c} |
| M-A2 | 23.4^{c} | 29.64^{c} | 7.94^{c} |
| M-B1 | 40.9^{b} | 32.86^{abc} | 11.65^{abc} |
| M-B2 | 42.5^{ab} | 32.86^{abc} | 11.17^{abc} |
| M-C1 | 46.5^{a} | 37.02^{a} | 13.28^{ab} |
| M-C2 | 46.9^{a} | 34.72^{a} | 14.68^{a} |
| M-D1 | 43.0^{ab} | 34.15^{ab} | 9.61^{abc} |
| M-D2 | 40.8^{b} | 32.82^{abc} | 9.47^{bc} |

| | Linear correlation analysis** | |
|---|---|---|
| Equation | y = 0.26 x + 23.05 | y = 0.22 x + 2.01 |
| R² coefficient | 0.85 | 0.70 |

*Means with different superscript letters in the same column are significantly different (P < 0.0001)
**Linear correlations were carried out between total BC concentration (% total protein) in soybean flours derived from different genotypes and inhibition values on lipid accumulation in adipocytes of SPH

SAH exerts an inhibitory effect on lipid accumulation in 3T3-L1 adipocytes and may affect lipid metabolism in 3T3-L1 adipocytes. Therefore, the effect of SPH on gene expression of enzymes, lipoprotein lipase (LPL) and de novo fatty acid synthesis (FAS), was evaluated. RNA was extracted from the treated 3T3-L1 adipocytes.

The following primers (SEQ ID NOs. 9-14) were used to amplify RNA sequences:

LPL:
Forward primer: 5′-CTGCTGGCGTAGCAGGAAGT-3′
Reverse primer: 5′-GCTGGAAAGTGCCTCCATTG-3′
FAS:
Forward primer: 5′-TCGGCGAGTCTATGCCACTATT-3′
Reverse primer: 5′-ACAGAGAACGGATGAGTTGTTCCT-3′
18S:
Forward primer: 5′-GATCCATTGGAGGGCAAGTCT-3′
Reverse primer: 5′-AACTGCAGCAACTTTAATATACGCTATT-3′

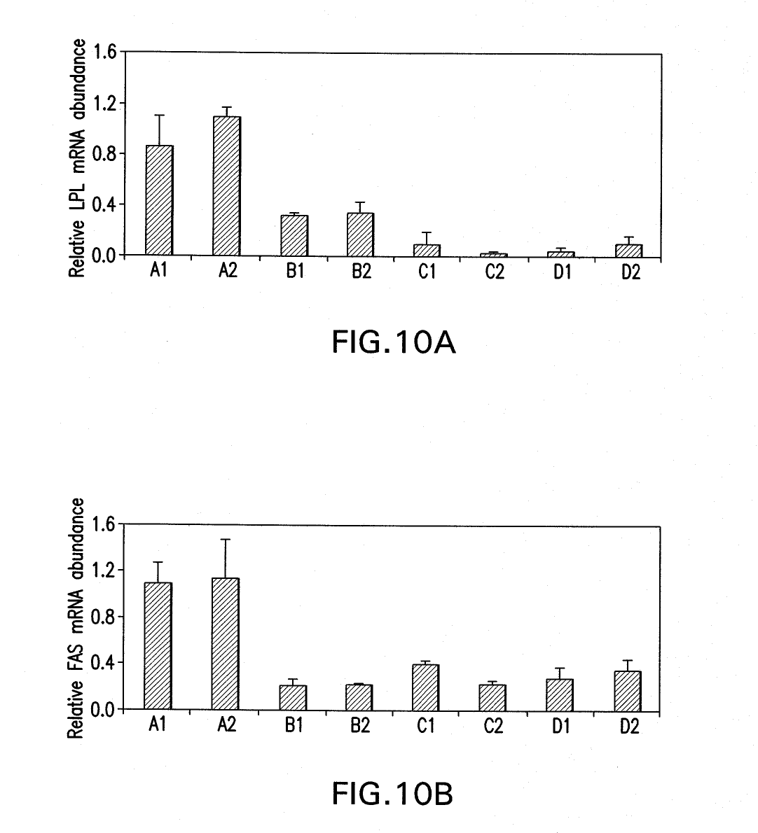

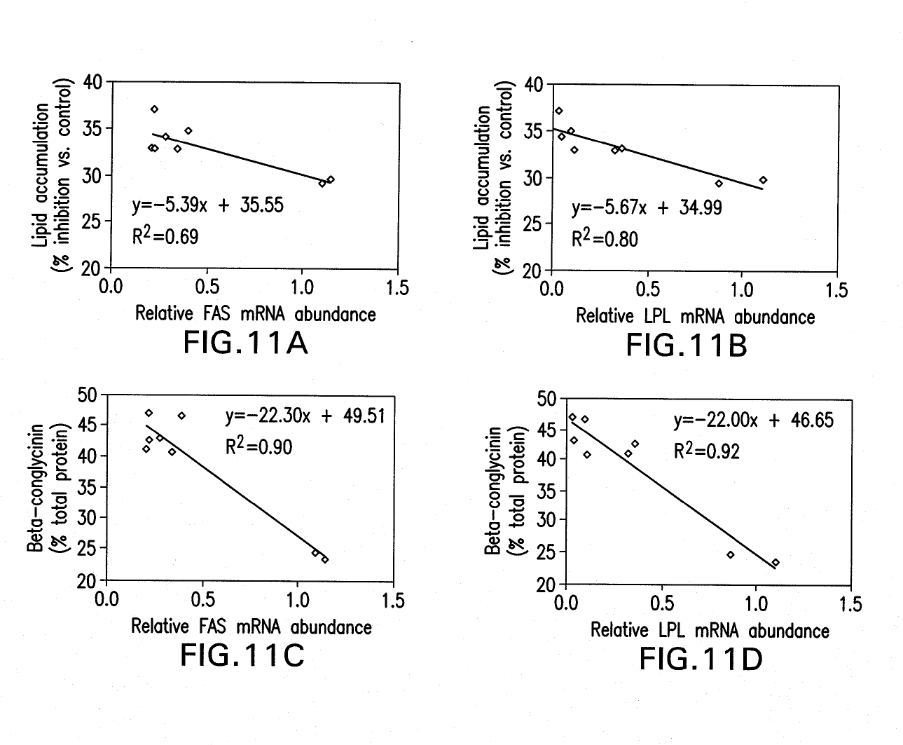

FIG. 10 shows the effect of SAH on LPL (A) and FAS (B) gene expression in 3T3-L1 adipocytes. SAH derived from 43% BC group showed a marked decrease of LPL and FAS mRNA abundance relative to the negative control (values <1); however, SAH from 24% BC did not affect LPL and FAS gene expression compared to negative control and LPL and FAS mRNA abundance was significantly lower compared to SAH from 43% BC group (P<0.0001) Inhibitory effect of alcalase SPH on lipid accumulation was negatively correlated with LPL (R²=−0.80) and FAS (R²=−0.69) mRNA relative abundance (FIGS. 11A and 11B). Moreover, BC concentration in soybean lines used to produced SAH was negatively correlated to LPL (R²=−0.90) and FAS (R²=−0.92) mRNA relative abundance (FIGS. 11C and 11D). Higher BC concentration in soybean lines down regulates of LPL and FAS gene expression in 3T3-L1 adipocytes.

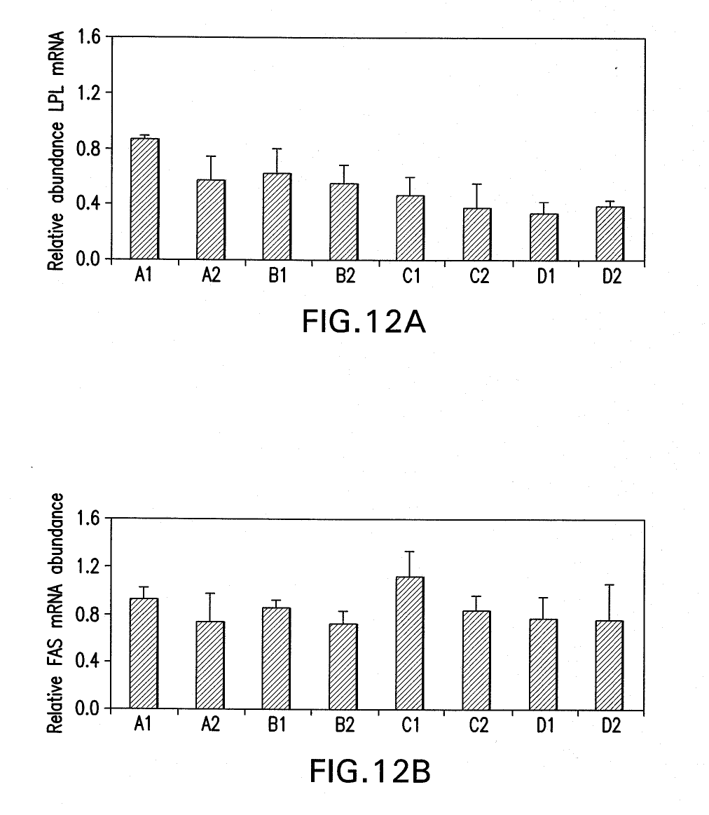

FIG. 12 shows the effect of SGIH on LPL (A) and FAS (B) gene expression in 3T3-L1 adipocytes. SGIH decreased LPL mRNA abundance compared to the negative control (values <1) and no significant differences were observed between SPH from 43% and 24% BC groups. On the contrary, SGIH did not change FAS mRNA abundance compared to negative control (values ≧1) and 24% BC group was not statistically different from 43% BC group (P>0.05). An inhibitory effect of SGIH on lipid accumulation was weakly correlated to LPL (R²=−0.3) mRNA relative abundance (data not shown). Furthermore, BC concentrations in soybean lines were also weakly correlated to LPL (R²=−0.5) mRNA relative abundance (data not shown). This suggested that SGIH may inhibit lipid accumulation by inhibiting LPL gene expression.

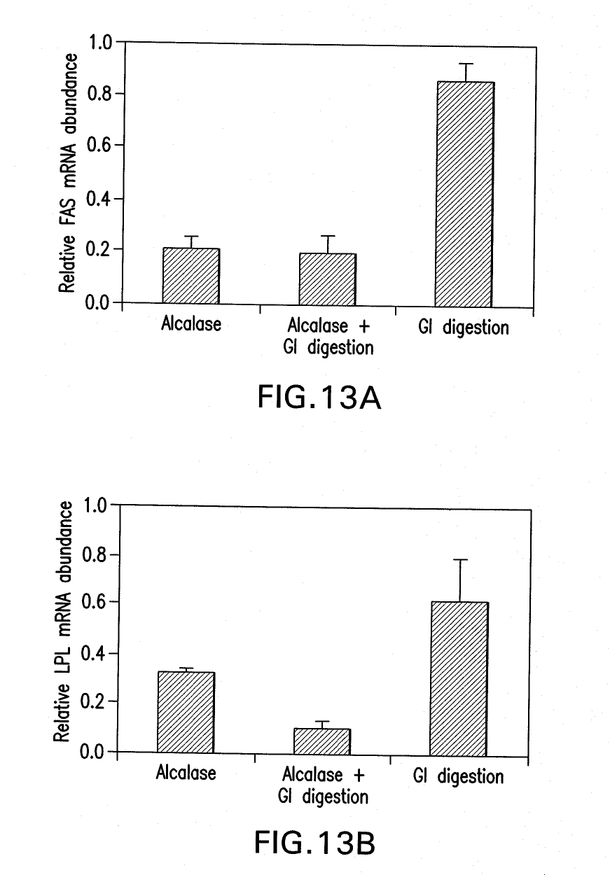

FIG. 13 shows the effect of SAH followed by simulated GI digestion and SGIH on LPL and FAS gene expression in 3T3-L1 adipocytes. Results showed that SAH retained their biological activity related to down regulation of LPL (P=0.0108) and FAS (P<0.0001) gene expression in 3T3-L1 adipocytes after simulated GI digestion. Moreover, it was observed a significantly higher inhibitory effect of SAH compared to SGIH on LPL (P=0.0108) and FAS (P<0.0001) gene expression in 3T3-L1 adipocytes. SAH (100 μM) from soybean lines enriched in β-conglycinin exerted an inhibitory effect on lipid accumulation by down regulating LPL and FAS gene expression in 3T3-L1 adipocytes.

Soybean lines lacking glycinin subunits (D1-D2) were selected as raw material to study the effect of SAH as potential anti-inflammatory agents. Macrophage cell line RAW 264.7 was cultured in growth medium containing DMEM, 1% penicillin/streptomycin, 1% sodium pyruvate and 10% FBS at 37° C. in 5% CO₂/95% air. The cell proliferation assay was conducted using the CellTiter 96® Aqueous One Solution Proliferation assay kit (Promega) using the novel tetrazolium compound 3-(4,5-dimethylthiazol-2-yl)-5-(3-carboxymethoxyphenyl)-2-(4-sulfophenyl)-2H-tetrazolium, inner salt (MTS), and an electron coupling reagent, phenazine ethosulfate (PES). Briefly 5×10⁴ cells were seeded in a 96-well plate and the total volume was adjusted to 200 μL with growth medium. The cells were allowed to grow for 24 h at 37° C. in 5% CO₂/95% air. After 24 h incubation, they were treated with different concentrations of SAH ranging from 0 to 25 μM for 24 h. After 24 h treatment, the growth medium was replaced by 100 μL fresh growth medium and 20 μL MTS/PES was added to each well. The plate was incubated for 2 h at 37° C. and the absorbance read at 515 nm. The percentage of viable cells was calculated with respect to cells treated with phosphate buffered saline as follows:

A_{treatment 515 nm}/A_{control 515 nm}*100=% cell viability

An examination of the cytotoxicity of SAH in RAW 264.7 macrophages by the MTS assay indicated that even the highest concentration tested (25 μM) did not affect the viability of the cells.

COX-2 and iNOS expression were determined in cell lysates. Treated cells were washed with ice cold DMEM and ice cold phosphate buffered saline before treatment with 200 μL Laemmli buffer with 5% β-mercaptoethanol lysis buffer. After lysis, the cell lysates were boiled for 5 min and approximately 25 μg of protein were loaded in 4-20% Tris-HCl ready gels for protein separation. The separated proteins were transferred to PVDF membranes and blocked with 5% non-fat dry milk in 0.1% Tris-buffered saline Tween® 20 (TBST) for 1 h at 4° C. After blocking, the membrane was washed with 0.1% TBST (5 times, 5 min each) and incubated with either COX-2 or iNOS mouse monoclonal antibodies (1:1000) at 4° C. overnight. The membrane was washed again and incubated with antimouse IgG horseradish peroxidase conjugate secondary antibody for 3-4 h at room temperature. After incubation and repeated washing, the expression of COX-2 and iNOS was visualized using chemiluminescent reagent following manufacturer's instructions.

Approximately 2×10⁵ cells were seeded in a 6-well plate and allowed to grow to its 80-90% confluency. The cells were treated with 1 μg/mL LPS with or without different concentrations of SPH dissolved in phosphate buffered saline ranging from 5 to 25 μM for 24 h. After 24 h treatment, the spent medium was collected and analyzed for nitric oxide and PGE₂. Nitrite accumulation, and indicator of NO synthesis, was measured in the culture medium by Griess reaction. Briefly, 100 μL of cell culture medium were plated in 96-well plate and an equal amount of Griess reagent constituted by 1% (w/v) sulfanilamide and 0.1% (w/v) N-1-(naphthyl)ethylenediamine-diHCl in 2.5% (v/v) H₃PO₄, was added. The plate was incubated for 5 min and the absorbance measured at 550 nm. The amount of nitric oxide was calculated using a sodium nitrite standard curve. For PGE₂ measurement, PGE₂ ELISA kit monoclonal was used following manufacturer's instructions.

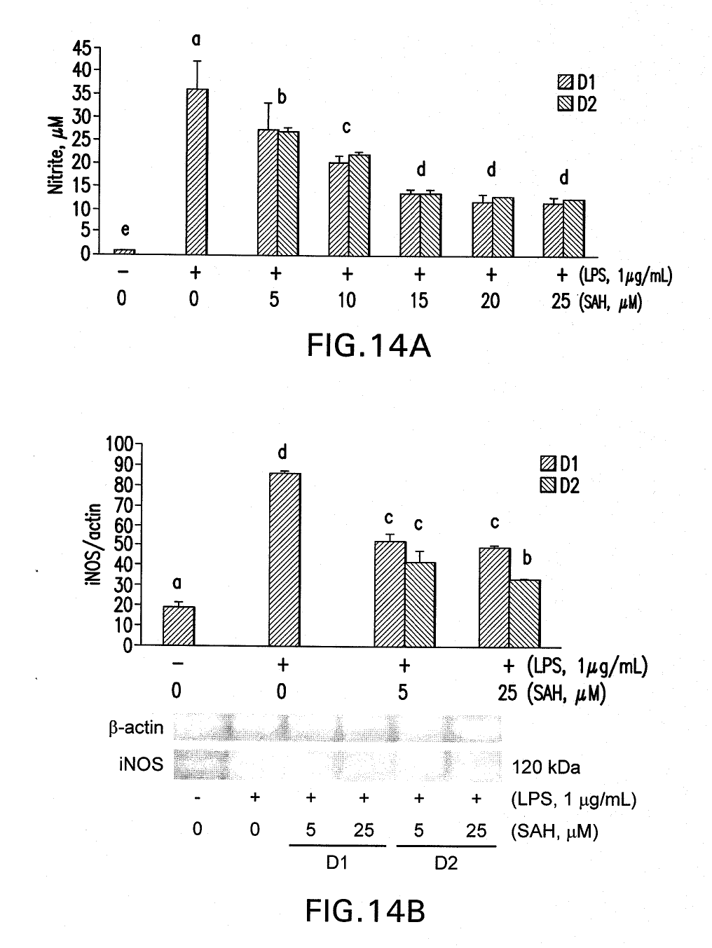

SAH D1 and D2 can inhibit production of the pro-inflammatory responses at low concentrations as 5 μM. The effect of SAH on NO production was determined by measuring the level of nitrite accumulation (the stable metabolite of NO) in culture media. LPS (1 μg/mL) induced significant nitrite production as compared with the negative control (P<0.0001). This was inhibited by SAH treatment in a dose dependent manner (P<0.0001) (FIG. 14A). The production of NO is controlled by iNOS. The expression of iNOS protein in SAH-treated RAW 264.7 cells was evaluated using western blot. The expression of the iNOS protein was barely detected in the non-stimulated cells (FIG. 14B). However, the level increased markedly 24 h after the LPS treatment. SAH exerted a significant inhibition of iNOS protein expression in the LPS-stimulated RAW 264.7 macrophages ((P<0.0001). Higher concentrations of SAH D2 (25 μM) resulted in a more marked inhibition of iNOS (P<0.0001).

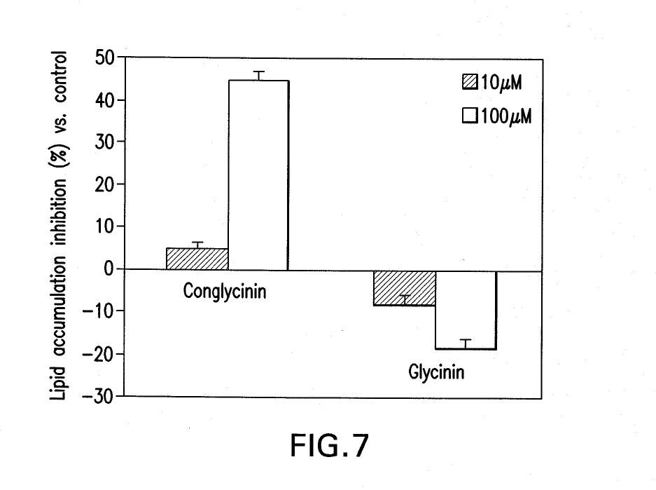

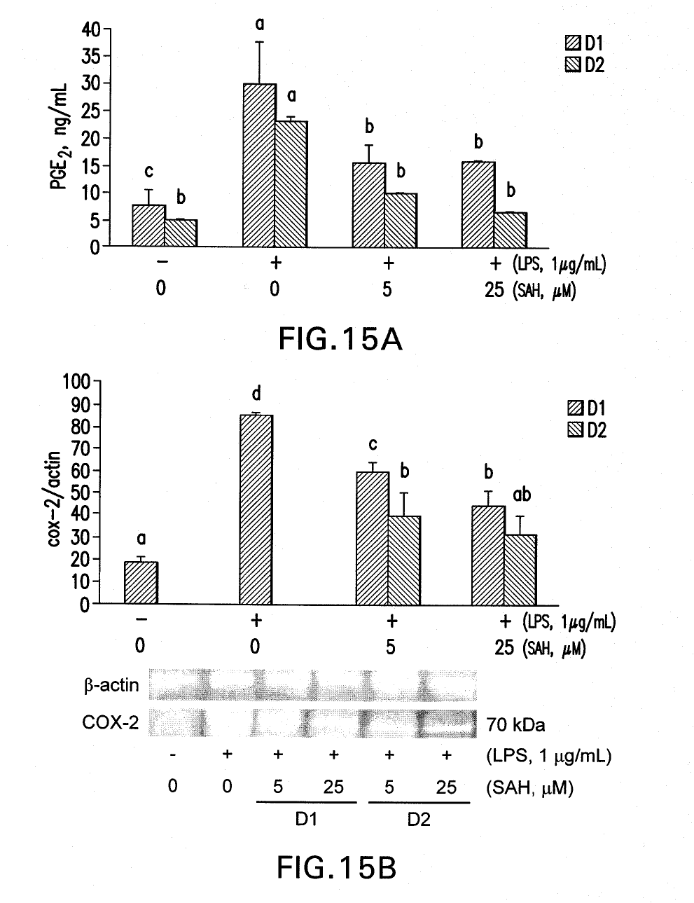

To further understand the role of SAH in inflammation, the production of prostaglandin E₂ (PGE₂) and expression of COX-2 on LPS-stimulated RAW 264.7 cells was measured. Western blot shows the expression of the COX-2 protein was significantly lower in the non-stimulated cells compared to LPS-stimulated RAW 264.7 macrophages 24 h after the LPS treatment (FIG. 15B). SAH effectively suppressed the PGE₂ production (P<0.0001) and COX-2 protein expression (P<0.0001) at 5 and 25 μM (FIGS. 7A and 7B). SAH D2 (5 μM) showed a significant higher reduction of PGE₂ (P<0.0001) and a higher inhibition of COX-2 protein expression (P<0.0001) than SAH D1 (FIG. 15A).

Treatment of macrophage with genotypes A2, B1 and C1 alcalase hydrolysates showed a significant reduction on the production of NO and PGE₂ and expression of iNOS and COX-2 when compared to the positive control. Nitric oxide production was inhibited from 18.1±0.9 to 35.7±2.5% while PGE₂ production was inhibited from 56.1±6.2 to 71.3±0.9%. This reduction was also accompanied by reduction of the inducible forms of nitric oxide synthase (31.2±0.6 to 53.5±2.6%) and cyclooxygenase (35.7±18.7 to 49.8±16.4%). Furthermore, at 5 μM concentration, no significant differences on the anti-inflammatory activities were observed when compared to hydrolysates from D1 and D2. These results indicated that low concentration of hydrolysates is not capable of differentiating among the 5 genotypes from each other with regards to their potential anti-inflammatory activity. The use of higher concentrations of the hydrolysates may be employed to further characterize the anti-inflammatory potential of these samples.

Soybeans enriched in β-conglycinins can provide hydrolysates that limit fat accumulation in fat cells and inflammatory pathways. Protein hydrolysates derived from soybeans containing high levels β-conglycinin, markedly inhibited lipid accumulation and inflammation indicators in adipocytes, in vitro. Furthermore, GI digestion of these hydrolysates did not cause a loss of bioactivity. SGIH inhibited lipid accumulation in a lesser extent by inhibiting LPL gene expression in 3T3-L1 adipocytes.

### Example 7

### Beneficial Compositions with Soy Protein and an Additional Plant-Sourced Component

Methods for formulating and testing compositions that comprise a protein-containing product, such as a soymeal, from a selected soybean, as well as another plant-derived component, are also contemplated. A soybean line or variety may be selected as described in the preceding Examples, for a beneficial bioactivity that enhances human health. Seed of such a soybean line may comprise total seed protein wherein at least 27-33% of the total seed protein is β-conglycinin. Alternatively, selection for a soybean line demonstrating a beneficial bioactivity may occur during the testing of the composition that is being prepared. Soymeal or other soy protein-containing product, including bioactive peptides, may be prepared from seed of such a variety, as also described above. Ground soybeans and one or more additional whole or ground plant tissue source(s) are heat treated in water and digested with one or more proteases to form a digest. The digest, or an extract of the digest, is included in a bioactivity assay to test for bioactivity associated with human health.

Composite bioactivity indices represent a novel way of utilizing findings from bioactivity measurements to develop an overall picture of the bioactivity of a product. A number of factors, such as elevated cholesterol and excessive body fat, can influence overall human health. The composite bioactivity index can be developed to include several bioactivities related to a targeted benefit such as weight management, or may include bioactivities related to several health benefits, such as anti-cancer, anti-lipid accumulation, and anti-inflammation, in the index. The identified health benefit factors are evaluated for activity and influence on overall human health. From the findings, an index and calculation of the index is developed. The index provides a powerful tool in evaluating and prioritizing products for human health effects. The bioactivity can be used on a number of products, such as soybean lines, soy meal, soy protein-containing products and other plant protein products.

The additional plant tissue source may be a protein or bioactive peptide source, and/or a source of another component such as of an oil, fiber, carbohydrate, or antioxidant, among others. The soymeal and the other plant-derived component source may be heat treated and/or digested together, or separately before the soy-derived and other plant-derived components are mixed for inclusion in the bioassay. The source of the non-soy plant-derived component may be a vegetable, such spinach or broccoli, among others.

### Example 8

### Soybean Varieties with Increased Antioxidant Capacity

The antioxidant capacity of soy protein hydrolysates prepared from seven soybean varieties (M-A2, M-B1, M-B2, M-C1, M-C2, M-D1, and M-D2) using Alcalase® (SAH) or simulated gastrointestinal digestion (SGIH) were evaluated. Total protein concentration of the hydrolysates were determined by DC Protein Quantification Assay using bovine serum albumin as standard curve (y=0.0002x−0.007, R²=0.99). Antioxidant capacity (AC) was determined by Oxygen Radical Absorbance Capacity (ORAC) assay (e.g. Cao et al., 1993). The reaction was measured by following the loss of fluorescence over time and comparing the area under the curves to a vitamin E analogue (Trolox) standard curve (y=0.3x+0.4, R²=0.99).

The protein concentration showed an average of 7.6±0.2 μg protein/μL for SAH (range: 5.2±0.2 to 10.2±0.1 μg protein/μL) and 2.6±0.1 μg protein/μL for SGIH (range: 1.9±0.1 to 3.1±0.1 μg protein/μL). The AC average value for SAH was 59.9±5.5 μM Trolox eq./μg protein (range: 52.8±0.3 to 69.4±14.3 μM Trolox eq./μg protein), whereas the AC average value for SGIH was 55.9±5.7 μM Trolox eq./μg protein (range: 46.6±6.4 to 69.7±4.9 μM Trolox eq./μg protein) (Table 9). Soybean glycinin concentrations negatively correlated with antioxidant capacities of soy protein hydrolysates. Furthermore, soybean beta-conglycinin was positively correlated with antioxidant capacities of soy protein hydrolysates. In addition, the digestion treatment, SAH or SGIH, did not appear to affect AC. Therefore a preferred soybean composition for preparing soy products for human health and also for products containing unsaturated fatty acids such as omega-3 fatty acids and CLA, are soybeans having less than 30% of the total protein as glycinins and greater than 27% of the proteins as beta-conglycinins

TABLE 9
Antioxidant activity of SAH and SGIH of seven soybean genotypes.

| | SGIH Treatment | | | | | SAH Treatment | | | | |
|---|---|---|---|---|---|---|---|---|---|---|
| | | | μM | | | | | μM | | |
| | | | Trolox | | | | | Trolox | | |
| Soybean | Beta | | eq./μg | Standard | | Beta | | eq./μg | Standard | |
| Genotype | Conglycinin | Glycinin | Protein | Deviation | CV | Conglycinin | Glycinin | Protein | Deviation | CV |
| M-A2 | 23.4 | 35.3 | 51.53 | 9.10 | 17.67 | 23.4 | 35.3 | 53.65 | 6.33 | 11.79 |
| M-B1 | 40.9 | 14.6 | 49.84 | 2.85 | 5.71 | — | — | — | — | — |
| M-B2 | 42.5 | 13.7 | 46.55 | 6.41 | 13.77 | 42.5 | 13.7 | 55.43 | 5.41 | 9.77 |
| M-C1 | 46.5 | 6.1 | 54.40 | 2.41 | 4.43 | 46.5 | 6.1 | 69.36 | 14.30 | 20.61 |
| M-C2 | 46.9 | 6 | 69.71 | 4.91 | 7.04 | 46.9 | 6 | 52.84 | 0.27 | 0.52 |
| M-D1 | — | — | — | — | — | 43 | 0 | 67.98 | 1.39 | 2.05 |
| M-D2 | 40.8 | 0 | 63.63 | 8.77 | 13.78 | — | — | — | — | — |

### Example 9

### Soybean Varieties Enhancing Microflora

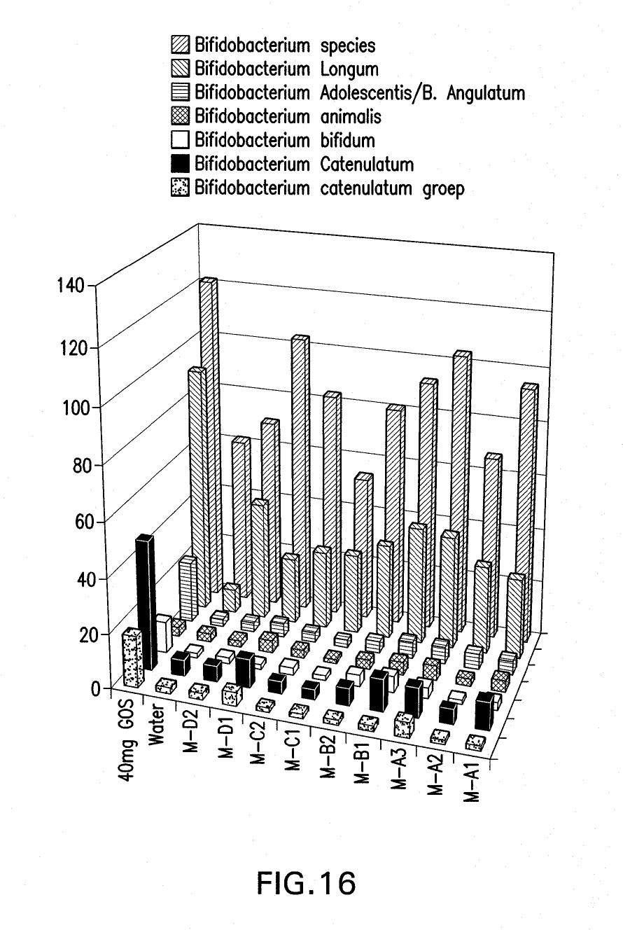

Analyses of human microbial flora (large intestinal flora from 8-10 individuals) following exposure to soy protein hydrolysates (1 mg peptides/mL) prepared from nine soy genotypes (M-A1, M-A2, A3, M-B1, M-B2, M-C1, M-C2, M-D1, and M-D2) using alcalase (SAH) were performed by TNO (Zeist, The Netherlands). The soy hydrolysates stimulated the growth of beneficial Bifidobacteria, but no significant differences were observed amongst the soy genotypes (FIG. 16).

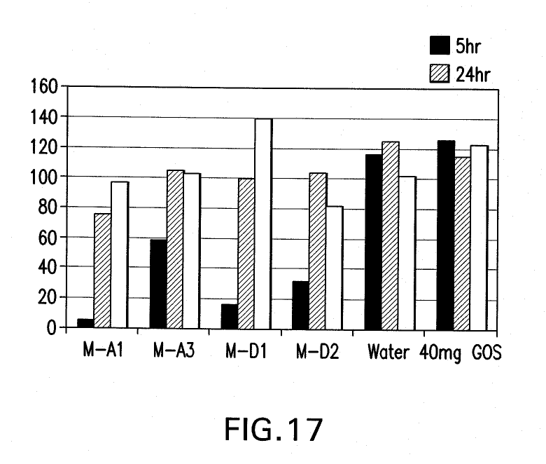

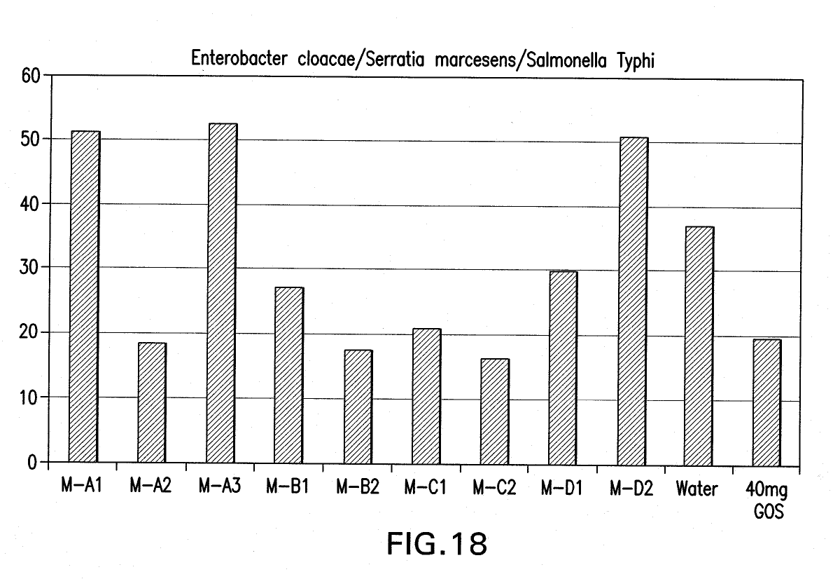

Enterobacteriaceae are a large family of bacteria that include bacteria that are responsible for a variety of human illnesses, including urinary tract infections, wound infections, gastroenteritis, meningitis, septicemia, and pneumonia. In the microflora analysis, soy hydrolysates appear to inactivate Enterobacteriaceae (FIG. 17). Soy hydrolysates M-A1, M-D1 and M-D2 had a significant lower signal for the Enterobacteriaceae after 5 hour of exposure to soy hydrolysates M-A1, M-D1 and M-D2. The effect was not observed for the positive or negative control. In addition, soy hydrolysates M-A2, M-B1, M-B2, M-C1, and M-C2 significantly reduced enterobacterial levels after 24 hrs of exposure (FIG. 18). The inhibitory effects of the hydrolysates on enterobacteria were consistent for the hydrolysates prepared from the B and C genotypes, unique in their higher concentrations of the α-subunit of β-conglycinin (17.7% to 20.8% of total protein compared to 10.4% to 13.7% of the total protein in genotypes A and D).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,992,375; U.S. Pat. No. 5,015,580; U.S. Pat. No. 5,024,944; U.S. Pat. No. 5,416,011; U.S. Pat. No. 5,525,305; U.S. Pat. No. 5,545,545; U.S. Pat. No. 5,637,785; U.S. Pat. No. 6,031,154; U.S. Pat. No. 6,140,085; U.S. Pat. No. 6,184,440; U.S. Pat. No. 6,486,383; U.S. Pat. No. 6,607,757; U.S. Pat. No. 6,774,284.
U.S. Patent Application Publication 2006/0246444; U.S. Patent Application Publication 2008/0113872; U.S. Patent Application Publication 2008/0096243.
Adams et al., J. Nutr., 134:511-516, 2004.
Agui et al. Peptide Science 195-198, 2005.
Allard, In: Principles of Plant Breeding, John Wiley & Sons, NY, 50-98, 1960.
Anderson et al., J. Nutr. 134:3011-3015, 2004.
Baba et al., J. Nutr. Sci. Vitaminol. (Tokyo), 50:26-31, 2004.
Beachy et al., Ann. Rev. Phytopathol. 28:451, 1990.
Beilinson et al., Theor. Appl. Genet., 104:1132-1140, 2002.
Boerma and Moradshahi, Crop Sci., 15:858-861, 1975.
Borthwick and Parker, Bot. Gaz., 100:374-387, 1938.
Brim and Stuber, Crop Sci., 13:528-530, 1973.
Cao et al., Free Radical Biol. Med. 14:303-311, 1993.
Charest et al., Plant Cell Rep. 8:643 1990.
Chen and Shoemaker, J. Hered., 89:211-215, 1998.
Chrispeels et al., J. Cell Biol., 93:306-313, 1982.
Christianson et al., Science, 222:632-634, 1983.
Comai et al., Nature 317:741-744 1985.
Criswell and Hume, Crop Sci., 12:657-660, 1972.
Diers et al., Theor. Appl. Genet., 89:297-304, 1993.
Duranti et al., J. Nutr., 134:1334-1339, 2004.
Eichholtz et al., Somatic Cell Mol. Genet. 13:67 1987.
Elliot et al., Plant Molec. Biol. 21:515 1993.
EP Appln. 0 242 246
EP Appln. 0640141
EP Appln. 0797673
Fehr, pp. 249-293 in: J. R. Wilcox, ed., Soybeans: Improvement, Production and Uses, Monograph No. 16, 2nd Edition, American Society of Agronomy, Madison, Wis., 1987a.
Fehr, In: Theory and Technique, and Crop Species Soybean, Iowa State Univ., Macmillan Pub. Co., NY, (1)(2):360-376, 1987b.
Fehr, in: Hybridization of Crop Plants, Fehr and Hadley (Eds.), Am. Soc. Agron. and Crop Sci. Soc. Am., Madison, Wis., 90-599, 1980.
Finer et al., In: Soybean: Genetics, Molecular Biology and Biotechnology, CAB Intl., Verma and Shoemaker (ed), Wallingford, Oxon, UK, 250-251, 1996.
Fisher et al., Plant Physiol., 102:1045-1046, 1993.
Fraley et al., Proc. Natl. Acad. Sci. USA, 80:4803, 1983.
Galvez et al. Cancer Res., 61:7473-7478, 2001.
Geiser et al., Gene, 48:109, 1986.
Gordon-Kamm et al., Plant Cell, 2:603-618, 1990.
Guha et al., Cellular Signaling, 13:85-94, 2001.
Hamner, in: Evans (ed.), The Induction of Flowering: Some Case Histories, Cornell Univ. Press, Ithaca, N.Y., 62-89, 1969.
Harada et al., Japan J. Breed., 33:23-30, 1983.
Hartweck et al., In Vitro Cell. Develop. Bio., 24:821-828, 1988.
Hotamisligil, Nature, 444:860-867, 2006.
Hurley et al. Can. J. Physiol. Pharmacol., 76:1000-10007, 1998.
Janderova et al., Obesity Res. 11:65-74, 2003.
Japan Pat Appln. 2005080533A2
Jing-Jing et al., Mol. Nutr. Food Res. 52, 631-645, 2008.
Jones et al., Science, 266:789, 1994.
Kakade et al., Anal. Biochem. 33 :255-258, 1970.
Kennedy et al., Cancer Res 56:679-82, 1996.
Knutzon et al., Proc. Natl. Acad. Sci. USA, 89:2624, 1992.
Kohno et al., J Atheroscler Thromb, 13: 247-255, 2006.
Krul et al., Carcinogenesis 23:1009-1016, 2002.
Kuhajda et al., Trends Pharmacol. Sci. 26:541-544, 2005.
Ladin et al., Plant Physiol., 84:35-41, 1987.
Lee et al., EMBO J., 7:1241, 1988.
Lee and Kim, Food Chem. 90:389-393, 2004.
Logemann et al., Bio/Technology, 10:305, 1992.
Mahmoud et al., J. Agric. Food Chem. 54:3916-3922, 2006.
Manzoni et al., J. Agric. Food Chem. 46:2481-2484, 1998.
Manzoni et al., J. Nutr. 133:2149-2155, 2003.
Marshall et al., Theor. Appl. Genet., 83:435, 1992.
Martin et al., Science, 262:1432, 1993.
Maruyama et al., J. Agric. Food Chem. 47:5278-5284, 1999.
Maruyama et al., JAOCS. 79:139, 2002.
Megias, J. Ag. Food Chem. 52:1928-1932, 2004.
Miki et al., Theor. Appl. Genet., 80:449, 1990.
Mindrinos et al., Cell, 78:1089, 1994.
Minekus et al., Appl. Microbiol. Biotechnol. 53:108-114, 1999.
Moriyama et al., Biosci. Biotechnol. Biochem., 68:352-359, 2004.
Nagano, et al., J. Agric. Food Chem. 44:3484-3488, 1996.
Nakamura et al., Soy Protein Res 7:13-19, 2004.
Nakamura et al., Soy Protein Res 8:1-7, 2005.
Nielsen et al., In: Cellular and molecular biology of plant seed development, Larkins and Vasil I K (Eds)., Kluwer Academic Publishers, Dordrecht, The Netherlands, 151-220, 1997.
Nielsen et al., Plant Cell., 1:313-328, 1989.
Nishi et al., J. Nutr., 133(2):352-357, 2003.
PCT Appln. US93/06487
PCT Appln. WO93/19181
PCT Appln. WO96/30517
Poehlman and Sleper, in: Breeding Field Crops, Iowa State University Press, Ames, 1995.
Przibila et al., Plant Cell 3:169, 1991.
Rahman et al., Proteomics 8, 569-581, 2008.
Rickert, et al. J. Food Sci. 69:303, 2004.
Salleh, et al. Biosci. Biotech. Biochem. 68:1091, 2004.
Schaafsma, JAOAC Int, 88:988-994, 2005.
Shah et al., Science, 233:478, 1986.
Shimomura et al. Genes Dev. 12, 3182-3194, 1998.
Shimoyamada et al., J. Ag. Food Chem. 46 :4793-4797, 1998.
Shanmugasundaram and Tsou, Crop Sci., 18:598-601, 1978.
Shen et al. J Appl Microbiol 102: 283-289, 2007.
Shibles et al., In: Crop Physiology, Some Case Histories, Evans (Ed.), Cambridge Univ. Press, Cambridge, England, 51-189, 1975.
Shiroza et al., J. Bacteriol., 170:810, 1988.
Simmonds, In: Principles of crop improvement, Longman, Inc., NY, 369-399, 1979.
Sneep and Hendriksen, In: Plant breeding perspectives, Wageningen, The Netherlands, Center for Agricultural Publishing and Documentation, 1979.
Søgaard et al., J. Biol. Chem., 268:22480-22484, 1993.
Stalker et al., Science, 242:419-423, 1988.
Steinmetz et al., Mol. Gen. Genet., 20:220, 1985.
Tezuka et al., J. Agric. Food Chem., 48 :1111-1117, 2000.
Tezuka et al., J. Agric. Food Chem., 52 :1693-1699, 2004.
Utsumi, in: Advances in Food and Nutrition Research, Kinsella (ed.), 36:89-208, Academic Press, San Diego, Calif., 1992.
Vanden Elzen et al., Plant Mol. Biol., 5:299, 1985.
Van Gaal et al. Nature 444, 875-880, 2006.
Van Nuenen et al., Microbiol. Ecol. Health Disease 15:137-144, 2003.
Wang et al., Compr. Rev. Food Sci. F. 4: 63-78, 2005.
Wang et al., J. Agric. Food Chem. 56:4012-4020, 2008.
Weisberg et al., J. Clin. Invest. 116, 115-124, 2006
Wold, H. “Partial Least Squares,” in S. Kotz and N. L. Johnson (Eds.), Encyclopedia of Statistical Sciences (Vol. 6), New York: Wiley, 581-591, 1985.
Wright et al., Plant Cell Reports, 5:150-154, 1986.
Zayas et al., Functionality of Proteins in Food, 1997.

Zuo et al., World J Gastroenterol 11: 5801-5806, 2005.

1. A method of selecting soybeans for human food with improved or standardized bioactivity associated with human health, wherein
a) ground soybeans are heat-treated in water and digested with one or more protease(s); and
b) the digest or extract of the digest is included in a bioactivity assay.

2. The method of claim 1, wherein the one or more protease(s) are selected from the group consisting of pepsin, pancreatin, and alcalase.

3. The method of claim 1, wherein the soybeans are dehulled soybeans, edamame or roasted soybeans.

4.-5. (canceled)

6. The method of claim 1, wherein
i) the ground soybeans are defatted prior to digestion;
ii) the heat-treatment prior to digestion is performed for from about 5 minutes to about 3 hours;
iii) the heat-treatment prior to digestion is performed at about 50° C. to about 85° C.:
iv) the heat-treatment prior to digestion is performed at about 50° C. for about 3 hours; or
v) the heat-treatment prior to digestion is performed at about 85° C. for about 5 minutes.

7.-9. (canceled)

10. The method of claim 1, wherein other enzymes are included such as lipase, alpha-amylase, trypsin and or chymotrypsin.

11. The method of claim 1, wherein
i) the digest is freeze-dried;
ii) the bioactivity assay comprises a chemical assay;
iii) the bioactivity assay comprises a chemical assay and antioxidant activity is determined.

12.-13. (canceled)

14. The method of claim 1, wherein the heat treatment prior to digestion is performed for from about 5 minutes to about 3 hours and wherein
i) bile acid binding is determined;
ii) a negative bioactivity is determined;
iii) a negative bioactivity is determined using the Ames assay;
iv) the bioactivity assay is a cell-based bioassay;
v) L1210 leukemia mouse line cells are used as a model to test soybeans for anticancer bioactivity;
vi) adipocyte cells are used as a model to test reduced lipid accumulation activity;
vii) macrophages are used as a model to test anti-inflammatory activity;
viii) the expression of mRNA for adiponectin is measured in adipocyte cells;
ix) expression of mRNA for LDL receptors and enzymes such as fatty acid synthetase and HMG CoA reductase is measured in liver cell lines;
x) expression of mRNA for enzymes such as fatty acid synthetase and lipoprotein lipase is measured in cell lines; or
xi) expression of mRNA for CCK, GLP-1, and PYY is determined in an enteroendocrine cell line.

15.-24. (canceled)

25. The method of claim 14 wherein the enteroendocrine cell line comprises STC-1 cells.

26. A soybean plant capable of producing seeds with enhanced bioactivity related to human and animal health.

27. A seed of the plant of claim 26, with enhanced bioactivity related to human and animal health wherein
a) the bioactivity comprises reduced cancer cell viability;
b) the bioactivity comprises reduced cancer cell viability and wherein the β-conglycinin content is greater than 27% of total protein content and the glycinin content is less than 30% of total protein content;
c) the bioactivity comprises increased LDL receptor activity;
d) the bioactivity comprises increased inhibition of lipid accumulation;
e) the bioactivity is increased adiponectin expression; or
f) the bioactivity is increased adiponectin expression and the β-conglycinin content is greater than 27% of total protein content and the glycinin content is less than 30% of total protein content.

28.-29. (canceled)

30. The soybean seed of claim 27, wherein the bioactivity comprises increased LDL receptor activity and wherein the β-conglycinin content is greater than 27% of total protein content and the glycinin content is less than 30% of total protein content.

31. The soybean seed of claim 30, wherein the Kunitz trypsin inhibitor content is less than 2% of the total protein content.

32. The soybean seed of claim 27, wherein the bioactivity comprises increased inhibition of lipid accumulation and wherein the β-conglycinin content is greater than 27% of total protein content and the glycinin content is less than 30% of total protein content.

33. (canceled)

34. The soybean seed of claim 32, wherein the Kunitz inhibitor content is less than 2% of the total protein.

35. The soybean seed of claim 27, wherein the bioactivity is reduced LPL or FAS expression.

36. The soybean seed of claim 35, wherein the β-conglycinin content is greater than 27% of total protein content and the glycinin content is less than 30% of total protein content.

37. The soybean seed of claim 27, wherein the bioactivity is increased antioxidant capacity.

38. The soybean seed of claim 37, wherein the β-conglycinin content is greater than 27% of total protein content and the glycinin content is less than 30% of total protein content.

39.-40. (canceled)

41. A method of producing a soybean plant with enhanced bioactivity for human and animal health, comprising:
a) screening at least two soybean seeds for an enhanced bioactivity for human or animal health;
b) selecting a seed having the enhanced bioactivity for human or animal health; and
c) growing a first plant from the selected seed.

42. The method of claim 41, further comprising the steps of:
d) crossing the first plant to a second soybean plant to form a population; and
e) selecting and growing at least one seed from the first or second plant having enhanced bioactivity for human or animal health.

43. A soybean protein product with enhanced bioactivity for human or animal health.

44. The product of claim 43, wherein
a) the β-conglycinin content is greater than 27% of total protein content and the glycinin content is less than 30% of total protein content;
b) the product is further defined as comprising an omega-3 fatty acid or a conjugated linoleic acid;
c) the product is further defined as comprising an omega 3-fatty acid and a conjugated linoleic acid; or
d) the product is selected from the group consisting of: extruded soybean, soybean meal, soyflour, defatted soyflour, soymilk, spray-dried soymilk, soy protein concentrate, texturized soy protein, hydrolyzed soy protein, soy protein isolates, and spray-dried tofu.

45.-47. (canceled)

48. A foodstuff made with the soybean protein product of claim 44, selected from the group consisting of: a pet food, a beverage, an infused food, a sauce, a soup, coffee creamer, a cookie, an emulsifying agent, bread, candy, an instant milk drink, gravy, noodles, soynut butter, soy coffee, roasted soybeans, a cracker, soymilk, tofu, tempeh, baked soybeans, a bakery ingredient, a bar, beverage powder, breakfast cereal, fruit juice, syrup, dessert, icing and fillings, a soft frozen product, a confection, and an intermediate food.

49. A method of inhibiting inflammation, pain or itching, comprising: applying to a subject a topical composition comprising β-conglycinin or a protease-treated preparation of β-conglycinin.

50. The method of claim 49 wherein the composition comprises:
a) a protease-treated preparation made from soybean seed wherein at least 27% of the total protein in the soybean seed comprises beta-conglycinins and less than 30% of the total protein in the soybean seed comprises glycinins; and
b) a cosmetically acceptable topical carrier.

51. A method of preventing or inhibiting inflammation and/or associated pain, comprising: consuming a food or drink made from soybean seed wherein at least 27% of the total protein in the soybean seed comprises beta-conglycinins and less than 30% of the total protein in the soybean seed comprises glycinins.

52. The method of claim 51 where the food is selected from the group consisting of: a liquid form of edamame; a powder form of edamame; roasted soybeans; soymilk; soy kefir; soy yogurt; soy flour; soy protein concentrate; soy protein isolate; and β-conglycinin isolate.

53. The method of claim 52 where the soymilk is whole bean soymilk.

54. The method of claim 53 where the soymilk is treated for over one hour with a protease.

55. The method of claim 54 where the protease is alcalase.

56. The method of claim 55 wherein consumption follows physical exercise or participation in a sporting competition.

57. The method of claim 53 wherein the β-conglycinin or β-conglycinin peptides is (are) administered to a subject in the amount of about 1 gram to about 50 grams per day.

58. A topical composition comprising β-conglycinin or a protease-treated preparation of β-conglycinin.

59. The composition of claim 58 wherein the composition comprises:
a) a protease-treated preparation made from soybean seed wherein at least 27% of the total protein in the soybean seed comprises β-conglycinins and less than 30% of the total protein in the soybean seed comprises glycinins; and
b) a cosmetically acceptable topical carrier.

60. The composition of claim 58, comprising β-conglycinin, wherein greater than 33% of the total soy protein or peptides originate from β-conglycinins.

61. A method of selecting soybeans and another plant protein source for human food with improved or standardized bioactivity associated with human health, wherein
a) ground soybeans and at least one additional whole plant component source or sources are heat-treated;
b) the heat treated preparation of step (a) is digested with one or more proteases to form a digest or an extract of the digest; and
c) the digest or extract of the digest is included in a bioactivity assay.

62. The method of claim 61 where the plant component source or sources are selected from the group consisting of: sunflower seeds, flax seeds, a nut, and a vegetable.

63. The method of claim 62, wherein the vegetable is spinach, broccoli, tomatoes, peppers, lettuce, purple carrot, bean, or cucumber.

64. A method of enhancing the growth of beneficial microflora in an animal gut comprising: consuming a food or drink comprising the product of claim 43.

65. The method of claim 64, wherein the animal is a human.
